SEQUENCE LISTING (12 sequences)

SEQ ID NO: 1 - protein, 273 aa, Hyphochytrium catenoides
GPMPFYDSRPPEGWPKGSINDMDYPLLGSICAVCCVFVAGSGIWMLYRLDLGMGYSCKPYKSGRAPEVNSLSGIICLLCGTMYAAKSFDFFDGGGTPFSLNWYWYLDYVFTCPLLILDFAFTLDLPHKIRYFFAVFLTLWCGVAAFVTPSAYRFAYYALGCCWFTPFALSLMRHVKERYLVYPPKCQRWLFWACVIFFGFWPMFPILFIFSWLGTGHISQQAFYIIHAFLDLTCKSIFGILMTVFRLELEEHTEVQGLPLNEPETLSLEVLFQ

SEQ ID NO: 2 - protein, 300 aa, Hyphochytrium catenoides
MPFYDSRPPEGWPRGSVNDMDYPLLGSICAISAIAIAGSGIWMLYRLDLGMGYSCKPYKSGRAPEVNSISGIVCLLCGTMYAAKSFDFFDGGGTPFSLNWYWYLDYVFTCPLLIVDFAFTLDIPQKLRYSIAVFVALWCAVAAFATPSAFRFAYYALGCCWFIPLSLFLIRDVKKRYQVYPPKCQRLLFWACVVFFGFWPLFPLLFIFSWQGSGHISRQAYYIIHAFLDLVCKSIFGFLMTFFRLELEEHTEVQGLPLKEPKVMDAAAKSRITSEGEYIPLDQIDINVGAPGSSLEVLFQ

SEQ ID NO: 3 - protein, 313 aa, Wobblia lunata
MSACPTPPTVVAPTDQSPNPNGGTTSCAQAAPSPQMSMNYTLLATIDGFSAVILLGSGVVFWYYSWRGTGFGAHPHPKRRPLELNTLSAAIAFLTGLMYVAKTSDFARDSTSEFSLNRYWYSDYIVTCPLIVMDLCFTVDVPYKLAFTGSTAVLLVIALASFIVRSPEKYIYFAIGCTLFSIFFYFFFNEVRKRIRYVPDCAKKDLERAMFIFFGFWPMFPVLWLLGFHTTGVISEEAFFIIHAFLDITCKTIFGVFMLRCRLHIEDYAWDELKRQRQEIDEENQKTLTAAGADGATVMMPVEEDDPYASGRT

SEQ ID NO: 4 - protein, 391 aa, Bilabria sp.
MSECCELCVCQKEPGTFGALIAVNTITAIILVAAGAYMAWKTAAGLGWNTRPHGPEGPPEENWLSPGISILCGVMYAFKAIDWASYNDTGESTAFSLNQVWYSDYLITCPLLVLDFCITVNLRYKLVFSSSIACLLAIAVSTFIVDAPYRYYMYGIGLAGFICAGYALWNEINAQREKIPDSAWWYLSAGRLIFFAGWPFFPLLWTLSFHTSGVINEEWYFILHAILDILCKAVFGFFMLGFRLELEELDFKAIEAEQAKLEGDKEAQALKNDKDTGEVLRHYNRASSGFFGQGIPDDQGSVSGGSVLMSRARRQHMLYRREASYLSMDPMAEKIRELEMLKKKIQKEVDSRSKKMQREMTARFAIQDDSDDEDGGRGAGSARRRKNARRG

SEQ ID NO: 5 - protein, 312 aa, Rhodamonas lens
GPMAHAPGTDQMFYVGTMDGWYLDTKLNSVAIGAHWSCFIVLTITTFYLGYESWTSRGPSKRTSFYAGYQEEQNLALFVNFFAMLSYFGKIVADTLGHNFGDVGPFIIGFGNYRYADYMLTCPMLVYDLLYQLRAPYRVSCSAIIFAILMSGVLAEFYAEGDPRLRNGAYAWYGFGCFWFIFAYSIVMSIVAKQYSRLAQLAQDTGAEHSLHVLKFAVFTFSMLWILFPLVWAICPRGFGWIDDNWTEVAHCVCDIVAKSCYGFALARFRKTYDEELFRLLEQLGHDEDEFQKLELDMRLSSNGERLEVLFQ

SEQ ID NO: 6 - protein, 333 aa, Chlamydomonas reinhardtii
STGSDATVPVATQDGPDYVFHRAHERMLFQTSYTLENNGSVICIPNNGQCFCLAWLKSNGTNAEKLAANILQWITFALSALCLMFYGYQTWKSTCGWEEIYVATIEMIKFIIEYFHEFDEPAVIYSSNGNKTVWLRYAEWLLTCPVILIHLSNLTGLANDYNKRTMGLLVSDIGTIVWGTTAALSKGYVRVIFFLMGLCYGIYTFFNAAKVYIEAYHTVPKGRCRQVVTGMAWLFFVSWGMFPILFILGPEGFGVLSVYGSTVGHTIIDLMSKNCWGLLGHYLRVLIHEHILIHGDIRKTTKLNIGGTEIEVETLVEDEAEAGAVSSEDLYFQ

SEQ ID NO: 7 - protein, 315 aa, Chlamydomonas reinhardtii
MDYGGALSAVGRELLFVTNPVVVNGSVLVPEDQCYCAGWIESRGTNGAQTASNVLQWLAAGFSILLLMFYAYQTWKSTCGWEEIYVCAIEMVKVILEFFFEFKNPSMLYLATGHRVQWLRYAEWLLTCPVILIHLSNLTGLSNDYSRRTMGLLVSDIGTIVWGATSAMATGYVKVIFFCLGLCYGANTFFHAAKAYIEGYHTVPKGRCRQVVTGMAWLFFVSWGMFPILFILGPEGFGVLSVYGSTVGHTIIDLMSKNCWGLLGHYLRVLIHEHILIHGDIRKTTKLNIGGTEIEVETLVEDEAEAGAVNKGTGK

SEQ ID NO: 8 - protein, 277 aa, Guillardia theta
ITCDPAIYGEWSRENQFCVEKSLITLDGIKYVQLVMAVVSACQVFFMVTRAPKVPWEAIYLPTTEMITYSLAFTGNGYIRVANGKYLPWARMASWLCTCPIMLGLVSNMALVKYKSIPLNPMMIAASSICTVFGITASVVLDPLHVWLYCFISSIFFIFEMVVAFAIFAITIHDFQTIGSPMSLKVVERLKLMRIVFYVSWMAYPILWSFSSTGACIMSENTSSVLYLLGDALCKNTYGILLWATTWGLLNGKWDRDYVKGRNVDGTLMPEYEQDLE

SEQ ID NO: 9 - protein, 228 aa, Halobacterium salinarum
TGRPEWIWLALGTALMGLGTLYFLVKGMGVSDPDAKKFYAITTLVPAIAFTMYLSMLLGYGLTMVPFGGEQNPIYWARYADWLFTTPLLLLDLALLVDADQGTILALVGADGIMIGTGLVGALTKVYSYRFVWWAISTAAMLYILYVLFFGFTSKAESMRPEVASTFKVLRNVTVVLWSAYPVVWLIGSEGAGIVPLNIETLLFMVLDVSAKVGFGLILLRSRAIFGE

SEQ ID NO: 10 - protein, 253 aa, Halobacterium salinarum
AVRENALLSSSLWVNVALAGIAILVFVYMGRTIRPGRPRLIWGATLMIPLVSISSYLGLLSGLTVGMIEMPAGHALAGEMVRSQWGRYLTWALSTPMILLALGLLADVDLGSLFTVIAADIGMCVTGLAAAMTTSALLFRWAFYAISCAFFVVVLSALVTDWAASASSAGTAEIFDTLRVLTVVLWLGYPIVWAVGVEGLALVQSVGATSWAYSVLDVFAKYVFAFILLRWVANNERTVAVAGQTLGTMSSDD

SEQ ID NO: 11 - protein, 290 aa, Dokdonia eikasta
MTQELGNANFENFIGATEGFSEIAYQFTSHILTLGYAVMLAGLLYFILTIKNVDKKFQMSNILSAVVMVSAFLLLYAQAQNWTSSFTFNEEVGRYFLDPSGDLFNNGYRYLNWLIDVPMLLFQILFVVSLTTSKFSSVRNQFWFSGAMMIITGYIGQFYEVSNLTAFLVWGAISSAFFFHILWVMKKVINEGKEGISPAGQKILSNIWILFLISWTLYPGAYLMPYLTGVDGFLYSEDGVMARQLVYTIADVSSKVIYGVLLGNLAITLSKNKELENLYFQSGSHHHHHH

SEQ ID NO: 12 - protein, 273 aa, Hyphochytrium catenoides
GPMPFYDSRPPEGWPKGSINDMDYPLLGSICAVCCVFVAGSGIWMLYRLDLGMGYSCKPYKSGRAPEVNSLSGIICLLCGTMYAAKSFDFFDGGGTPFSLNWYWYLDYVFTCPLLILDFAFTLDLPHKIRYFFAVFLTLWCGVAAFVTPSAYRFAYYALGCCWFTPFALSLMRHVKERYLVYPPKCQRWLFWACVIFFGFWPMFPILFIFSWLGTGHISQQAFYIIFAFLDLTCKSIFGILMTVFRLELEEHTEVQGLPLNEPETLSLEVLFQ

(19) United States
(12) Patent Application Publication
(10) Pub. No.: US 2026/0337252 A1
(43) Pub. Date: Oct. 29, 2026

(54) Enhanced Light Gated Potassium Selective Channelrhodopsin

(71) Applicants: The Board of Trustees of the Leland Stanford Junior University, Stanford, CA (US); The University of Tokyo, Bunkyo-ku, Tokyo (JP)

(72) Inventors: Karl A. Deisseroth, Stanford, CA (US); Yoon Seok Kim, Redwood City, CA (US); Charu Ramakrishnan, San Jose, CA (US); Hideaki Kato, Bunkyo-ku, Tokyo (JP); Seiya Tajima, Bunkyo-ku, Tokyo (JP)

(21) Appl. No.: 19/116,934

(22) PCT Filed: Oct. 27, 2023
(86) PCT No.: PCT/US2023/078107

§ 371 Date: Mar. 28, 2025

Related U.S. Application Data
(60) Provisional application No. 63/420,371, filed on Oct. 28, 2022.

(51) Int. Cl. C07K 14/435; C12N 13/00; C12N 15/85

(52) U.S. Cl. CPC C07K 14/79144; C12N 13/00; C12N 15/85

(57) ABSTRACT

Disclosed herein include light gated potassium selective channelrhodop sins (KCRs). Various embodiments include a KCR comprising an amino acid substitution that alters a property of the KCR. Certain embodiments are derived from a natural KCR, including one or more KCRs from Hyphochytrium catenoides. Additional embodiments are directed to nucleic acids encoding a KCR. Additional embodiments include a vector and/or a cell containing a nucleic acid encoding a KCR. Further embodiments include methods for expressing a KCR and/or optogenetically controlling a cell via the KCR.

### CROSS-REFERENCE TO RELATED APPLICATIONS

This application claims the benefit of U.S. Provisional Patent Application No. 63/420,371, filed Oct. 28, 2022, entitled “Enhanced Light Gated Potassium Selective Channelrhodopsin” to Deisseroth, et al., the disclosure of which is hereby incorporated by reference herein in its entirety.

### SEQUENCE LISTING

This application hereby incorporates by reference the material of the electronic Sequence Listing filed concurrently herewith. The material in the electronic Sequence Listing is submitted as an xml (.xml) file entitled “STAN-2048WO_Seq_List.xml” created on Oct. 26, 2023, which has a file size of approximately 15 kilobytes and is herein incorporated by reference in its entirety.

### INTRODUCTION

Optogenetics has emerged as a powerful and versatile tool for studying and modulating biological systems. By enabling precise and reversible control over cellular functions through the use of light, this technology has revolutionized the understanding of complex biological processes. The significance of optogenetics lies in its ability to unravel cellular mechanisms, advance neuroscience, and pave the way for therapeutics.

Optogenetics typically utilizes A) light-sensitive proteins, which can respond to specific wavelengths of light and allow for the activation or inhibition of cellular processes; B) versatile expression systems to deliver light sensitive proteins to a cell; C) specialized light delivery systems to deliver light with special and/or temporal precision; and D) enhanced experimental models that incorporate optogenetics into complex in vitro setups. Given the diversity of biological processes, there is a need in the field to develop additional light-sensitive proteins to elucidate additional pathways.

### SUMMARY

Disclosed herein include light gated potassium selective channelrhodopsins (KCRs). Various embodiments include a KCR comprising an amino acid substitution that alters a property of the KCR. Certain embodiments are derived from a natural KCR, including one or more KCRs from Hyphochytrium catenoides. Additional embodiments are directed to nucleic acids encoding a KCR. Additional embodiments include a vector and/or a cell containing a nucleic acid encoding a KCR. Further embodiments include methods for expressing a KCR and/or optogenetically controlling a cell via the KCR.

### BRIEF DESCRIPTION OF THE FIGURES

FIG. 1 provides a schematic of an experimental procedure of ATR-FTIR spectroscopy either ion perfusion (top) or light illumination (bottom) systems.

FIGS. 2A-2I provide exemplary representations of cryo-EM densities of HcKCR1 and HcKCR2. FSC-weighted sharpened maps were calculated by cryoSPARC v3.2.0 for HcKCR1 and cryoSPARC v3.3.2 for HcKCR2, respectively. Transmembrane helices for HcKCR1 (FIG. 2A) and HcKCR2 (FIG. 2B). Retinal binding pocket (FIG. 2C), the Schiff base region (FIG. 2D), K84 (FIG. 2E), ECL1 (FIG. 2F), lipid molecule (FIG. 2G), the N-terminal region (FIG. 2H) of HcKCR2. (FIG. 2I) Two different rotamers observed in F144 of HcKCR1.

FIGS. 3A-3B provide exemplary data of HPLC analysis of the chromophore configuration of HcKCR1 WT. (FIG. 3A) Representative HPLC profiles of the chromophore of HcKCR1 under dark (top) and light conditions (bottom). Abbreviations “at”, “9”, “11”, and “13” indicate the peaks of all-trans, 9-cis, 11-cis, and 13-cis retinal oximes, respectively. (FIG. 3B) Calculated composition of retinal isomers in HcKCR1 under dark and light conditions. Data are presented as mean±SEM (n=3). Values are listed in the table. Green light (510±5 nm) was used for illumination. Light adaptation was achieved by illumination for 1 min followed by incubation in the dark for 2 min.

FIG. 4A provides an exemplary cryo-EM density map (left) and ribbon representation of the HcKCR1 homotrimer viewed parallel to the membrane (middle) and viewed from the intracellular side (right), colored by protomer (blue, magenta, and purple), retinal (yellow), and lipid (grey), respectively. Grey bars indicate approximate location of the lipid bilayer.

FIG. 4B provides an exemplary cryo-EM density map (left) and ribbon representation of HcKCR2 homotrimer viewed parallel to the membrane (middle) and viewed from the intracellular side (right), colored by protomer (orange, green, and red), retinal (purple), and lipid (grey), respectively. Grey bars indicate approximate location of the lipid bilayer.

FIGS. 4C-4D provide exemplary monomeric structures of HcKCR1 (FIG. 4C) and HcKCR2 (FIG. 4D). 7-TM domains of HcKCR1 and HcKCR2 are colored in blue and orange, respectively. Retinal and ECL1 are colored in yellow for HcKCR1 and purple for HcKCR2, respectively.

FIGS. 4E-4G provide exemplary structural comparisons among HcKCR1, HcKCR2, ChRmine, and C1C2. HcKCR1 (blue) superimposed onto HcKCR2 (orange) (FIG. 4E), ChRmine (red) (FIG. 4F), and C1C2 (green) (FIG. 4G) from different angles. Retinal and ECL1 are colored in yellow (HcKCR1), purple (HcKCR2), green (ChRmine), and pink (C1C2). TMs 4-6 are displayed with transparency for clarity. Compared to ChRmine, TM1 and the cytoplasmic half of TM7 of HcKCR1 are tilted by about 7 and 10 degrees, respectively.

FIG. 5A provides exemplary schematics of Schiff base regions of HcKCR1 (left), HcKCR2 (middle), and ChRmine (right). Water molecules are represented by spheres. The black dashed lines indicate H-bonds.

FIG. 5B provides exemplary absorption spectra of HcKCR1 (left) and HcKCR2 (right) at pH 7.5. The traces of WT, D105N, and D229N are colored in blue, red, and green, respectively. The λmax values are shown above each trace.

FIGS. 6A-6B provide exemplary pH-titrated absorption spectra of HcKCR1 and HcKCR2. (FIG. 6A) The absorption spectra of HcKCR1 WT (left), D105N (middle), and D229N (right) from pH 2.2 to 11.0. (FIG. 6B) The absorption spectra of HcKCR2 WT (left), D105N (middle), and D229N (right) from pH 2.2 to 11.0. The λmax value at each pH is listed in the table.

FIG. 7A provides exemplary photocurrent amplitudes of WT, D105N and, D229N of HcKCR1 (left) and HcKCR2 (right), respectively. Mean±SEM (n=4-10); Kruskal-Wallis test with Dunnett's test. **p<0.01.

FIGS. 7B-7C provide exemplary voltage-clamp traces of HcKCR1 WT and 27 mutants (FIG. 7B) and HcKCR2 WT and 8 mutants (FIG. 7C), collected from −96 mV to +4 mV in steps of 10 mV (for C110T mutants, traces are collected from −96, −56, and −16 mV). HEK293 cells were recorded while stimulated by 1 s of 0.7 mW mm-2 irradiance at 560 nm for HcKCR1 and 470 nm for HcKCR2.

FIG. 8A provides exemplary time-series traces of absorption change for HcKCR1 WT and D105N mutant at specific wavelength. For HcKCR1 WT, the probe wavelength at 617 nm (light red), 480 nm (light green), 384 nm (light purple), and 404 nm (light blue) corresponds to K, L and N, M1, and M2 intermediates, respectively. The corresponding wavelengths of K, L and N, M′, and M″ intermediates for HcKCR1 D105N are 609 nm (red), 515 nm (green), 378 nm (purple), and 394 nm (blue), respectively. The cyan line represents the absorption changes of pyranine monitored at 454 nm.

FIG. 8B provides exemplary transient photocurrent changes of HcKCR1 induced by pulsed flash laser. Green and yellow lines indicate the raw trace and the fitting curve, respectively.

FIG. 8C provides exemplary yellow photocycle schemes of HcKCR1 WT (left) and D105N mutant (right) determined by flash photolysis experiment shown in FIG. 8B.

FIG. 8D provides exemplary transient absorption spectra of HcKCR1 WT (left) and D105N (right).

FIG. 8E provides exemplary absorption spectra of the initial state (gray), K/L1 (red), L2/M1 (orange), M2/N1 (green), N2 (light blue), and HcKCR1′ of HcKCR1 WT (left), and those of the initial state (gray), K/L1 (red), L2/M′ (orange), M″/N1 (green), N2 (light blue), and HcKCR1′ of D105N (right). The spectra are calculated from the decay-associated spectra of transient absorption changes shown in FIGS. 8A and 8D.

FIG. 9A provides exemplary schematics of retinal binding pockets of HcKCR1 (left), HcKCR2 (middle), and ChRmine (right). Residues forming the retinal binding pockets are shown in stick model form.

FIG. 9B provides exemplary sequence alignment for residues in the retinal binding pocket.

FIG. 9C provides exemplary peak photocurrent amplitudes of WT and four mutants of HcKCR1 (top) and HcKCR2, respectively (bottom). Mean±SEM (n=3-11); Kruskal-Wallis test with Dunnett's test.

FIG. 9D provides exemplary data for τ_{off} of WT and four mutants of HcKCR1 (top) and HcKCR2 (bottom), respectively. Mean±SEM (n=3-11); Kruskal-Wallis test with Dunnett's test. **p<0.01.

FIGS. 9E-9F provide exemplary data for photocurrent (FIG. 9E) and τ_{off} of channel closing (FIG. 9F). Mutants are categorized by location: intracellular vestibule or internal constriction site (IV/ICS) vs. extracellular vestibule or extracellular constriction site (EV/ECS). Sample size (number of cells) indicated in parentheses. Data are mean±S.E.M (n=3-11); one-way ANOVA followed by Dunnett's test. *p<0.05, **p<0.01, ***p<0.001, and ****p<0.0001.

FIG. 10A provides exemplary schematics of retinal β-ionone ring in ChRmine (PDB: 7W9W), C1C2 (PDB: 3UG9), HsBR (PDB: 5ZIM), and C1C2GA mutant (PDB: 4YZI).

FIGS. 10B-10C provides exemplary schematics of cryo-EM densities around the β-ionone ring of HcKCR2. Blue and green/red maps are FSC-weighted sharpened map calculated by cryoSPARC v3.3.2, and Fo-Fc maps calculated by the program Servalcat, respectively. All-trans (FIG. 10B) and 6-s-cis (FIG. 10C) retinal are modeled against the FSC-weighted sharpened map. Positive (green) and negative (red) Fo-Fc difference density pairing (±5.2 σ, where σ is the standard deviation within the mask) is observed between C18 and A136 (top), suggesting rotation of the β-ionone ring.

FIG. 11A provides exemplary schematics of β-ionone rings of HcKCR1 and HcKCR2 (top), and chemical structures of all-trans and 6-s-cis retinal (bottom). Red lines represent C5-C6-C7-C8 bonds.

FIG. 11B provides exemplary absorption spectra of HcKCR1 and 2 WT and their swapping mutants (T136A/G140A for HcKCR1 and A136T/A140G for HcKCR2). The λₘₐₓ values are shown above each trace.

FIG. 12 provides exemplary schematics of ion-conducting pathways in C1C2 (PDB: 3UG9), CrChR2 (PDB: 6EID), GtACR1 (PDB: 6CSM), HsBR (PDB: 5ZIM), and KR2 (PDB: 3X3C). Key residues for K+ selectivity in HcKCRs are shown as stick models. Intra- and extracellular cavities are calculated with the program HOLLOW.

FIG. 13A provides an exemplary comparison of ion-conducting cavities between HcKCR1 (left), HcKCR2 (middle), and ChRmine (right). TMs 4-6 are displayed with higher transparency. The residues located along the cavities are shown in stick model form. Intra- and extracellular cavities are calculated with the program HOLLOW. The black dashed rectangles indicate the IV and EV regions highlighted in (FIG. 13B) and (FIG. 13C), respectively. The black dashed lines and arrows represent H-bonds and the putative ion-conducting pathway, respectively. Locations of ICS, CCS, and ECS are indicated at the left side of each panel.

FIGS. 13B-13C provide exemplary schematics of IV (FIG. 13B) and EV (FIG. 13C) of HcKCR1 (left), HcKCR2 (middle), and ChRmine (right). Cavities are calculated with the program HOLLOW, and the black dashed lines indicate H-bonds. Locations of ICS, CCS, and ECS are indicated at the left side of each panel.

FIGS. 14A-14B provide exemplary patch clamp characterization of HcKCR1 and HcKCR2. (FIG. 14A) Residues along the ion-conducting cavities in HcKCR1 WT. Residues where mutation significantly changes Erev are highlighted in magenta. (FIG. 14B) Eᵣₑᵥ summary for mutations of the residues shown in (FIG. 14A). Mutants are categorized as the mutants of EV or ECS (left) and IV or ICS (right). Mean±SEM (n=3-17); one-way ANOVA with Dunnett's test. *p<0.05 **p<0.01,***p<0.001,****p<0.0001.

FIGS. 14C-14D provide exemplary schematics of the selectivity filter region of HcKCR1 WT (cryo-EM structure, left), Y222A mutant (homology model, middle), and W102Q mutant (homology model, right), viewed parallel to the membrane (FIG. 14C) and viewed from the extracellular side (FIG. 14D). Cavities are calculated with the program HOLLOW, and the black dashed lines indicate the closest distance between atoms of adjacent amino acids.

FIG. 14E provides exemplary patch clamp characterization of HcKCR1 and HcKCR2 under physiological and reversed ion balance conditions. At left: ion concentrations for voltage-clamp recordings. Erev summary for WT and three mutants of HcKCR1 and HcKCR2 under physiological (middle) or reversed (right) conditions. Mean±SEM (n=4-21); one-way ANOVA with Dunnett's test. **p<0.01 ****p<0.0001.

FIG. 15 provides exemplary voltage-clamp traces of HcKCR1 WT and 3 mutants in physiological (top) and reversed (bottom) conditions. Traces are collected from −124 mV to +16 mV in steps of 10 mV for the physiological condition and from −26 mV to +104 mV in steps of 10 mV for the reversed condition. HEK293 cells were recorded while stimulated with 1 s of 0.7 mW mm⁻² irradiance at 560 nm.

FIG. 16 provides exemplary ATR-FTIR difference spectra upon exchange of NaCl/KCl (top), NaCl/NaBr (middle) for HcKCR1 in dark (left) and light (right) conditions. The spectra for KR2 are shown in the bottom as the reference. Unlike the case with KR2 and KcsA, the flat spectra of HcKCR1 indicate that K+ does not stably bind to HcKCR1 in either dark or light conditions.

FIG. 17 provides an exemplary schematic of architecture of the representative prokaryotic K+ channel, KcsA (PDB: 1K4C). Transmembrane topology (left). Each subunit contains two TMs with a short loop containing the K+ selectivity filter. The tetrameric assembly viewed from the extracellular side and viewed parallel to the membrane (middle), colored by protomer (blue, green, red, and orange). The ion-conducting cavity is colored in semitransparent grey. K+ ions and the TVGYG motif are depicted by ball and stick models, respectively. Magnified view of the selectivity filter (right). Only two subunits are shown for clarity.

FIG. 18A provides exemplary traces from molecular dynamics (MD) simulation of HcKCR1 WT (top) and the D116N mutant (bottom); distances between D/N116 and R224 (green), and between D/N116 and K⁺ (magenta), are plotted for each monomer in a trimer. The gray shaded region at the beginning of the simulation marks the equilibration period during which the protein was restrained to the cryo-EM conformation.

FIG. 18B provides exemplary superposition of the HcKCR1 cryo-EM structure and the MD simulation snapshot. The purple sphere indicates K+. Pink upward and downward arrows represent the flipping movements of R244 and the entry of K⁺ to the binding site, respectively.

FIG. 18C provides an exemplary MD simulation snapshot showing the transient binding of partially dehydrated K⁺.

FIG. 18D provides exemplary ionic and hydration radii of sodium (Na⁺), potassium (K⁺), and guanidinium (Gu⁺) ions.

FIG. 18E provides exemplary current-voltage (1-V) relationships of HcKCR1 WT (left) and D116N mutant (right) in the presence and absence of GuHCl in the intracellular solution. Mean±SEM (n=3-8).

FIG. 19A provides an exemplary model for the K⁺ selectivity in KCRs when the concentrations of extracellular Na⁺ and intracellular K⁺ are high (physiological/normal condition), permeation of large, hydrated cations such as Na⁺ and Ca²⁺ is blocked at the size filter formed by W102, F/Y221, Y222, and H225. In contrast, K⁺ can enter the pore (under physiological ion balance conditions chiefly from the intracellular side, when the interaction between D116 and R244 is broken); K⁺ can become partially dehydrated, permeate through the ion-conducting pathway, and pass the size filter for release to the extracellular space.

FIG. 19B provides an exemplary model for the K+ selectivity in KCRs when the concentrations of intracellular Na⁺ and extracellular K⁺ are high (reversed condition), Na⁺ can move outward, just as K⁺ moves outward under physiological conditions. Inward K⁺ currents are possible in this condition through the aromatic size-exclusion filter at the EV (the size of hydrated K⁺ is smaller than that of Na⁺ or Ca²⁺). TMs 1, 2, 4-6 are removed for clarity. Black and pink arrows indicate the cation flow and the conformational change of R244, respectively. K⁺, Na⁺, Ca²⁺, and oxygen and hydrogen atoms of water molecules are shown as spheres colored in purple, green, brown, red and small white, respectively. Magenta circles represent the K⁺ selective size filters.

FIG. 20 provides an exemplary structure-based amino acid sequence alignment of microbial rhodopsins in accordance with various embodiments (SEQ ID NOs:1-11, respectively).

### DEFINITIONS

As used herein, the term “nucleic acid” refers to a polymeric form of nucleotides of any length, including nucleic acids that range from 2-100 nucleotides in length and nucleic acids that are greater than 50 nucleotides in length. The terms “nucleotide” refers to a sugar, a base, and a phosphate group. The terms “nucleobase” and “base” are used interchangeably herein. The term “nucleic acid” includes polymers of canonical (adenine (A), cytosine (C), guanine (G), thymine (T), uracil (U)) and non-canonical bases, chemically or biochemically modified or derivatized nucleotides, and nucleotides having modified sugar-phosphate backbones in which the conventional backbone has been replaced with non-naturally occurring or synthetic backbones. Conventional backbones are generally considered to be a ribose-phosphate backbone (as used in ribonucleic acid (RNA)) and a deoxyribose-phosphate backbone (as used in deoxyribonucleic acid (DNA)). Non-naturally occurring, synthetic, or otherwise non-conventional backbones, including replacing a ribose or deoxyribose with another sugar (e.g., threose), a peptide, or other moiety. Examples of non-naturally occurring, synthetic, or otherwise non-conventional backbones include xeno nucleic acid (XNA), peptide nucleic acid (PNA), morpholino nucleic acid, locked nucleic acid (LNA), glycol nucleic acid (GNA), 1,5-anhydrohexitol nucleic acid (HNA), Cyclohexene nucleic acid (CeNA), Fluoro Arabino nucleic acid (FANA), and threose nucleic acid (TNA). Certain nucleic acids may contain one or more nucleotides with a non-conventional backbone amongst conventional backbones—for example, 1 or more nucleotides may be LNA nucleotides, while the remaining nucleotides are DNA nucleotides. A nucleic acid may be of any convenient length, e.g., 2 or more nucleotides, such as 4 or more nucleotides, 10 or more nucleotides, 20 or more nucleotides, 50 or more nucleotides, 100 or more nucleotides, 300 or more nucleotides, such as up to 500 or 1000 or more nucleotides.

As used herein, the term “plurality” contains at least 2 members. In certain cases, a plurality may have 5 or more, such as 6 or more, 7 or more, 8 or more, 9 or more, 10 or more, 20 or more, 30 or more, 40 or more, 50 or more, 60 or more, 70 or more, 80 or more, 90 or more, 100 or more, 300 or more, 1000 or more, 3000 or more, 10,000 or more, 100,000 or more members.

Numeric ranges are inclusive of the numbers defining the range.

The percent identity of two nucleotide sequences can be determined by aligning the sequences for optimal comparison purposes (e.g., gaps can be introduced in the sequence of a first sequence for optimal alignment). The nucleotides at corresponding positions are then compared, and the percent identity between the two sequences is a function of the number of identical positions shared by the sequences (i.e., % identity=#of identical positions/total #of positions×100). When a position in one sequence is occupied by the same nucleotide as the corresponding position in the other sequence, then the molecules are identical at that position. A non-limiting example of such a mathematical algorithm is described in Karlin et al., Proc. Natl. Acad. Sci. USA 90:5873-5877 (1993). Such an algorithm is incorporated into the NBLAST and XBLAST programs (version 2.0) as described in Altschul et al., Nucleic Acids Res. 25:389-3402 (1997). When utilizing BLAST and Gapped BLAST programs, the default parameters of the respective programs (e.g., NBLAST) can be used. In one aspect, parameters for sequence comparison can be set at score=100, wordlength=12, or can be varied (e.g., wordlength=5 or wordlength=20).

As used herein, the term “light gated” refers to the utilization of light (typically in the form of photons) to control or modulate the operation of a system, process, or device. This can include various applications such as optical switches, sensors, detectors, or any technology that employs light to trigger specific actions or reactions.

As used herein, the term “excitation maximum” and “absorption maximum” refer to a peak absorption wavelength associated with a moiety. If such moieties are fluorescent (i.e., also emit a photon or other radiation), “excitation maximum” and “absorption maximum” may be used interchangeably.

The methods described herein may include multiple steps. Each step may be performed after a predetermined amount of time has elapsed between steps, as desired. As such, the time between performing each step may be 1 second or more, 10 seconds or more, 30 seconds or more, 60 seconds or more, 5 minutes or more, 10 minutes or more, 60 minutes or more and including 5 hours or more. In certain embodiments, each subsequent step is performed immediately after completion of the previous step. In other embodiments, a step may be performed after an incubation or waiting time after completion of the previous step, e.g., a few minutes to an overnight waiting time.

As used herein, the terms “evaluating”, “determining,” “measuring,” and “assessing,” and “assaying” are used interchangeably and include both quantitative and qualitative determinations.

### DETAILED DESCRIPTION

Before the methods and systems of the present disclosure are described in greater detail, it is to be understood that the methods and systems are not limited to particular embodiments described, as such may, of course, vary. It is also to be understood that the terminology used herein is for the purpose of describing particular embodiments only, and is not intended to be limiting, since the scope of the methods and systems will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the methods and systems. The upper and lower limits of these smaller ranges may independently be included in the smaller ranges and are also encompassed within the methods and systems, subject to any specifically excluded limit in the stated range. Where the stated range includes one or both of the limits, ranges excluding either or both of those included limits are also included in the methods and systems.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which the methods and systems belong. Although any methods and systems similar or equivalent to those described herein can also be used in the practice or testing of the methods and systems, representative illustrative methods and systems are now described.

All publications and patents cited in this specification are herein incorporated by reference as if each individual publication or patent were specifically and individually indicated to be incorporated by reference and are incorporated herein by reference to disclose and describe the materials and/or methods in connection with which the publications are cited. The citation of any publication is for its disclosure prior to the filing date and should not be construed as an admission that the present methods and systems are not entitled to antedate such publication, as the date of publication provided may be different from the actual publication date which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the methods and systems, which are, for clarity, described in the context of separate embodiments, may also be provided in combination in a single embodiment. Conversely, various features of the methods and systems, which are, for brevity, described in the context of a single embodiment, may also be provided separately or in any suitable sub-combination. All combinations of the embodiments are specifically embraced by the present disclosure and are disclosed herein just as if each and every combination was individually and explicitly disclosed, to the extent that such combinations embrace operable processes and/or compositions. In addition, all sub-combinations listed in the embodiments describing such variables are also specifically embraced by the present methods and systems and are disclosed herein just as if each and every such sub-combination was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present methods. Any recited method can be carried out in the order of events recited or in any other order that is logically possible.

### Potassium Selective Channelrhodopsins

Channelrhodopsins (ChRs) exist in various forms, such as non-selective channelrhodopsins that are specific to anions (i.e., anion channelrhodopsins, ACRs) and/or cations (i.e., cation channelrhodopsins, CCRs). While CCRs and ACRs have been used to stimulate and inhibit neuronal activity, respectively, the non-selective nature and unpredictability of these proteins can limit their application. Additionally, because repolarization of neuronal membranes universally occurs via an efflux of K⁺ ions, potassium selective channelrhodopsins (KCRs) have been sought after and would be ideal for optogenetic applications involving neuronal stimulation.

Many embodiments are directed to KCRs. In certain instances, the KCR is derived from a pump-like channelrhodopsin (PLCR). PLCRs are channelrhodopsins that possess a greater sequence similarity to a pump-type rhodopsin than to canonical channelrhodopsins. PLCRs can operate as ion channels. KCRs and/or PLCRs of various embodiments can be derived from extant channelrhodopsins. Such KCRs can be isolated from microbes, including protists. Certain embodiments are derived from a hyphochytrid protist, such as species from the Canteriomyces genus, Cystochytrium genus, Hyphochytrium genus, Latrostrium genus, Reessia genus, and/or Rhizidiomyces genus. Some embodiments are a channelrhodopsin derived from one or more of Hyphochytrium catenoides, H. elongatum, H. hydrodictyi, H. infestans, H. oceanum, and H. peniliae. Some preferred embodiments are a channelrhodopsin derived from H. catenoides. Certain embodiments are derived from Hyphochytrium catenoides Kalium channelrhodopsin 1 (HcKCR1; SEQ ID NO: 1) and/or Hyphochytrium catenoides Kalium channelrhodopsin 2 (HcKCR2; SEQ ID NO: 2).

Like many rhodopsins, many embodiments are formed as an oligomeric assembly of subunits. These subunits can be the same monomeric subunit or different monomeric subunits, such that the oligomeric assembly is homomeric or heteromeric, respectively. The oligomeric assembly can be formed of any number of monomeric subunits to be functional, such as 2 subunits (i.e., dimer), 3 subunits (i.e., trimer), 4 subunits (i.e., tetramer), 5 subunits (i.e., pentamer), 6 subunits (i.e., hexamer), 7 subunits (i.e., heptamer), 8 subunits (i.e., octamer), 9 subunits (i.e., nonamer), 10 subunits (i.e., decamer), or more. Some select embodiments are formed as a dimer, a trimer, a pentamer, or a hexamer.

Each monomeric subunit can be formed with at least one transmembrane domain. The at least one transmembrane domain from each subunit can form part of a central pore allowing transfer of an ion (e.g., potassium, K⁺) across a cellular membrane. In additional instances, a transmembrane domain can anchor the subunit (and/or protein as a whole) to a membrane. In various instances, the at least one transmembrane domain is 2 transmembrane domains, 3 transmembrane domains, 4 transmembrane domains, 5 transmembrane domains, 6 transmembrane domains, 7 transmembrane domains, 8 transmembrane domains, 9 transmembrane domains, 10 transmembrane domains, or more. In some preferred embodiments, the at least one transmembrane domain comprises 7 transmembrane domains. In various instances, the at least one transmembrane domain is a helix (e.g., alpha helix). In some preferred instances, the at least one transmembrane domain comprises a seven-helix transmembrane (7TM) domain.

Further embodiments include a chromophore attached to the at least one transmembrane domain. When excited, such chromophores can induce a structural change in the protein to allow ion transfer (e.g., K⁺ transfer) across a membrane. Such structural changes can include isomerization of the at least one transmembrane domain, such as isomerization from a cis to a trans configuration (or vice versa) of a linkage (or bond) within a transmembrane domain. In certain instances, the isomerization can be a single linkage or at multiple linkages (including all possible linkages). In various instances, the chromophore is linked to the at least one covalent bond. In certain instances, the bond is a Schiff base linkage.

In certain instances, the chromophore is not-specific to a wavelength of light (e.g., can absorb light at all wavelengths), while in some instances, the chromophore is wavelength specific (e.g., possesses an absorption maximum at a particular wavelength). Particular wavelengths can include light in the visible, ultraviolet (UV), and/or infrared (IR) spectra. In certain instances, the chromophore is responsive to UV light (e.g., ~100 nm to ~400 nm), visible light (e.g., ~400 nm to ~800 nm), and/or IR light (e.g., ~800 nm to ~1 mm). Each of these spectra can be further subdivided, such that a chromophore may be specific to UV-A (e.g., ~315 nm to ~400 nm), UV-B (e.g., ~280 nm to ~315 nm), UV-C (e.g., ~100 nm to ~280 nm), IR-A (e.g., ~800 nm to ~1400 nm), IR-B (e.g., ~1400 nm to ~3 μm), IR-C (e.g., ~3 μm to ~1 mm). Visible light can further be divided by color wavelengths, such as violet (e.g., ~400 nm ~450 nm), blue (e.g., ~450 nm to 485 nm, cyan (e.g., ~485 nm to ~500 nm), green (e.g., ~500 nm to ~565 nm), yellow (e.g., ~565 nm to ~590 nm), orange (e.g., ~590 nm to ~625 nm), and/or red (e.g., ~625 nm to ~800 nm).

Certain embodiments are derived from a native protein in that they may contain at least one amino acid substitution. In certain instances, the amino acid substitution can alter a property of the KCR. Such properties can include one or more of ion selectivity, channel kinetics, light absorption wavelength, and/or any relevant property. Ion selectivity can include a preference or selectivity for a single ion, class of ions, and/or ambivalence toward ions (e.g., non-selective).

Certain instances are selective for a specific ion, such as an ion selected from H⁺, Na⁺, K⁺, Li⁺, Ag⁺, Cu⁺, Ca²⁺, Mg²⁺, Zn²⁺, Fe²⁺, Cu²⁺, Ni²⁺, Cd²⁺, Pb²⁺, Al³⁺, Fe³⁺, Cl⁻, Br⁻, I⁻, O²⁻, S²⁻, and N³⁻. Certain embodiments are non-selective between certain ions, including (but not limited to) non-selective between Na⁺ and K⁺, non-selective between monovalent cations (e.g., H⁺, Na⁺, K⁺, etc.), non-selective between divalent cations (e.g., Ca²⁺, Mg²⁺, Zn²⁺, etc.), non-selective between monovalent and divalent cations (e.g., H⁺, Na⁺, K⁺, Ca²⁺, Mg²⁺, Zn²⁺, etc.), non-selective between trivalent cations (e.g., ⁺, Pb²⁺, Al³⁺, Fe³⁺, etc.), non-selective between cations (e.g., H⁺, Na⁺, K⁺, Li⁺, Ag⁺, Cu⁺, Ca²⁺, Mg²⁺, Zn²⁺, Fe²⁺, Cu²⁺, Ni²⁺, Cd²⁺, Pb²⁺, Al³⁺, Fe³⁺, etc.). Additional embodiments can be non-selective for similar groupings of anions, including monovalent, divalent, trivalent, etc.

In certain instances, the at least one amino acid substitution alters the channel kinetics, where channel kinetics are generally considered to be the alteration of channel activation, isomerization, and/or any other property. In some instances, the channel kinetics can be the time to switch on (τₒₙ) and/or switch off (τ_{off}) a KCR. By altering a time on or a time off, various embodiments can increase or decrease responsiveness and/or limit photobleaching for a similar amount of activation. For example, shortening a τₒₙ or τ_{off} can allow for more rapid excitation cycles or more specific patterning without bleed-through from a previous activation. However, lengthening τ_{off} may prevent photobleaching or the inability to activate a KCR of an embodiment. For example, a KCR may lose light sensitivity with prolonged exposure from a light; a longer τ_{off} can allow a KCR to be active for a longer time with a single excitation of the KCR, thus prolonging the ability to use a KCR.

In certain instances, the at least one amino acid substitution alters the absorption spectrum of the chromophore. The light wavelength can be altered to increase or decrease specificity for a wavelength, where specificity is generally considered the range of wavelengths that can activate the chromophore. For example, an amino acid substitution may shift the absorption maximum to a higher or lower wavelength and/or broaden or narrow the absorption spectrum distribution. As a non-limiting illustration, the absorption maximum may shift from approximately 565 nm to approximately 665 nm (or vice versa). A non-limiting illustration of altering the absorption spectrum distribution can be where one standard deviation from approximately 100 nm to 200 nm (or vice versa).

In various instances, the amino acid substitution can be selected from one or more of the following: W102Q, D105N, Y106W, T109A, C110T, D116N, T136A, A136T, G140A, A140G, F221A, Y222A, Y222F, H225F, H225A, H225Y, and D229N. In certain instances, embodiments with one or both of W102Q and Y222A may be non-selective for Na⁺ and K⁺, while F221A, H225F, H225A, and H225Y may increase K⁺ selectivity, and Y22F may lead to an intermediate specificity between the two previous scenarios (e.g., intermediate between non-selective for Na⁺ and K⁺ and increased K⁺ selectivity). Additionally, certain embodiments may use Y106W and/or T109A to decrease τ_{off}, while C110T may increase τ_{off}. Certain embodiments may alter the absorption spectra (e.g., absorption maximum, absorption spectrum, etc.), including one or more of D1 05N, T136A, A136T, G140A, A140G, and/or D229N.

One of skill in the art will understand methods to augment, alter, or otherwise vary an amino sequence and assess its efficacy. As such, certain embodiments may possess an amino acid sequence having at least 70%, 75%, 80%, 85%, 90%, 95%, 96%, 97%, 98%, or 99% identify to one or both of HcKCR1 (SEQ ID NO: 1) and HcKCR2 (SEQ ID NO: 2).

In should be noted that the various options or alternatives described above, including particular sources of a KCR, specific KCRs, and amino acid substitutions to alter selectivity, wavelength, and/or kinetics and specific examples of such substitutions can be used individually or in combination with each other, such that certain embodiments may possess multiple substitutions to both alter light absorption and channel kinetics. Any other combination may further be used, (e.g., channel kinetics and selectivity, absorption and selectivity, etc.). Some select embodiments may be derived from HcKCR1 or HcKCR2 and the amino acid substitution is selected from one or more of C110T, Y222A, and H225F. As a non-limiting example, the H225F substitution of HcKCR1 is provided as SEQ ID NO: 12.

### Nucleic Acids

Various embodiments are directed to nucleic acids that encode a KCR as described herein. Nucleic acid embodiments can comprise a coding sequence that encodes a KCR as described herein. The coding sequence can include one or more exons and 0 or more introns.

Such coding sequences can be codon optimized for a particular species, where codon optimization utilizes preferred codon sequences for a particular species. Additional embodiments may include additional features that can assist in expressing a KCR, replicating the nucleic acid, and/or any other applicable function, including (but not limited to) a promoter, a signal peptide sequences, a polyadenylation (PolyA) signal sequence, a terminator, a translational regulatory sequence (e.g., a ribosome binding site and/or an internal ribosome entry site (IRES)), an enhancer, a silencer, an insulator, a boundary element, a replication origin, a matrix attachment site, a post-transcriptional regulatory element (PRE), an upstream enhancer (USE), and/or a locus control region. Such promoters can be selected from (but not limited to), a species-specific promoter, an inducible promoter, a tissue-specific promoter, and/or a cell cycle-specific promoter. (See e.g., Parr et al., Nat. Med.3:1145-9 (1997); the contents of which are herein incorporated by reference in their entirety.)

Certain nucleic acids may include multiple coding sequences. Such as sequences that can be used as a selectable marker, including an antibiotic resistance gene, a fluorescent marker, and/or other selectable marker. Certain instances may further encode for a second KCR, such as described herein. A second coding sequence may be under the control of the same promoter as the first KCR/coding sequence and/or under an independent promoter. As a non-limiting example, selectable markers may be under control of a constitutive promoter, while a KCR may be under control of an inducible and/or tissue-specific promoter. In certain embodiments multiple genes (e.g., KCRs, selectable markers, etc.) may be in a single open reading frame and under the control of a single promoter. In such instances, these genes can be separated by a self-cleaving 2A peptide, and IRES sequence, and/or any other relevant separator or translational initiator.

Such nucleic acids may be linear, circular, single stranded, and/or double stranded as appropriate for a particular purpose, host species, etc. For example, certain nucleic acids are circular (e.g. plasmid, BAC, etc.), which can be used for replication and/or preservation within a bacterial vector. Certain embodiments may be linear, such as for transfection into a cell and/or integration into chromosomal DNA. Single-stranded embodiments may include RNA embodiments that are used for transient expression. Certain instances of single-stranded nucleic acids may be DNA-based, such as for use in certain viral vectors.

Nucleic acids of various embodiments can be manufactured via various applicable methodologies. In certain instances, solid-phase synthesis is used, where a chemically modified solid support, usually controlled-pore glass, is functionalized with the first nucleotide, typically protected at its reactive sites. Then, nucleotides are added one at a time in a stepwise manner. Each nucleotide addition involves deprotection, coupling, and washing steps. Such methodologies can be automated, allowing precise control of the sequence and efficient production of such nucleic acids. Additional embodiments use enzymes (e.g., DNA polymerases and reverse transcriptases) to synthesize nucleic acids. For example, reverse transcription is a process that uses reverse transcriptase to synthesize complementary DNA (cDNA) from an RNA template. Polymerase chain reaction (PCR) is a well-known method for DNA amplification, creating numerous copies of a specific DNA sequence using a DNA polymerase enzyme. Large fragment synthesis can be manufactured by assembling shorter oligonucleotide sequences. This process can be achieved through various methods, such as ligation, polymerase cycling assembly (PCA), or isothermal assembly. Overlapping sequences are designed to ensure proper assembly. Once manufactured or assembled, nucleic acids of such embodiments may further be purified, assessed for quality, quantified, and/or any other relevant process. Purification can be used to remove impurities (e.g., salts, solvents, incomplete and/or truncated molecules, etc.). Purification can be via any applicable technique, such as high-performance liquid chromatography (HPLC), ultrafiltration, size-exclusion chromatography, and/or another form of purification. Similarly quality and/or quantity can be assessed via spectroscopy, electrophoresis, and/or any other applicable methods that can assess quality and/or quantity of a manufactured nucleic acid of embodiments described herein.

Certain embodiments are directed to a vector that includes a nucleic acid as described herein. A vector may refer to a nucleic acid including additional sequences that assist in the function of a KCR coding sequence and/or an organism or cell that includes a nucleic acid that can be used for a particular function. A nucleic acid vector in accordance with many embodiments may be selected from a plasmid, a cosmid, a virion, a viroid, a virus, a BAC, a YAC, and/or any other nucleic acid construct. Organismal vectors may include a virus, a bacterium, a virion, a viroid, and/or other type of cell that includes the nucleic acid as described herein. Such vectors can be used for transfection (e.g., viral, bacterial, etc.), replication, expression, and/or preservation. Transfection vectors can include viruses, such as adenovirus, adeno-associated virus (AAV), lentivirus, vaccinia virus, vaculovirus, herpes simplex virus (HSV), alphavirus, sendai virus, picornavirus, and/or any other virus capable of transfecting an appropriate cell (e.g., neuron) with a nucleic acid as described herein. Certain embodiments may be cloned into bacteria (e.g., Escherichia coli), yeast (e.g., Saccharomyces cerevisiae), and/or bacteriophage for replication and/or preservation, such as in a library of particular molecules.

### Methods of Transfection and Optogenetic Control

Additional embodiments are directed to methods of transfecting a cell with a nucleic acid encoding a KCR as described herein. In certain instances, transfection occurs by inserting a nucleic acid or nucleic acid vector into a cell for expression and/or a biological vector (e.g., a bacterium or a virus). The cell for expression can be a cell where there is a desire for optogenetic control. Such cells of interest can be muscle cells, neurons, epithelial cells, blood cells, fibroblasts, immune cells, endothelial cells, and/or any other type of cell. As noted above, such potassium flux can be helpful for studying neuronal function; thus some preferred embodiments are directed to a cell comprising a nucleic acid as described herein. In additional preferred embodiments are directed to a neuron comprising a nucleic acid as described herein.

In certain instances, the method may include contacting a cell with a nucleic acid or a vector, both of which are described in the previous section. In various instances, contacting a cell comprises transfecting the cell or otherwise introducing the nucleic acid or vector into the cell for expression. In certain instances, transfection uses a biological system and/or a mechanical system. Mechanical systems can include particle bombardment (i.e., biolistics), heat shock, electroporation, and/or any other method of introducing a nucleic acid into a cell that does not require a biological vector. Methods and systems for mechanical transfection can be found in WO 2023/136932, US 2019/0136224, and/or US 2018/0142248; the disclosures of which are hereby incorporated by reference in their entireties.

Transfection can also use biological methodologies, such as viral-mediated and/or bacterial-mediated transformation, such as using vectors as described previously. In such instances, the biological systems can introduce the nucleic acid for stable expression (such as by genome integration) or transient expression, where the nucleic acid is expressed until it is fully degraded or otherwise expires. Biological processes for biological transfection are disclosed in one or more of U.S. Pat. Nos. 9,719,107, 7,803,622, US 2017/0183673, US 2015/0111955, US 2011/0209251, WO 2009/122962; the disclosures of which are hereby incorporated by reference in their entireties.

Once a cell is contacted with the nucleic acid and/or vector, certain embodiments may verify the presence of the nucleic acid or vector within the cell. Many ways exist to test for the transfection, including nucleic acid amplification, use of the selectable marker (e.g., antibiotic presence), fluorescence, ELISA, and/or any other method that can indicate a presence of the nucleic in the cell.

As noted above, various cells are subject to optogenetic control. As mentioned previously, optogenetics uses a light stimulation to activate a cellular function. In various embodiments, the method comprises illuminating cell with a light source. In many instances, the light source comprises a wavelength that includes the absorbance maximum of the KCR. In many instances, the illumination of the cell expressing the KCR triggers a conformational change in the KCR allowing for transport of an ion across the membrane. The transport of the ion can stimulate and/or inhibit a cellular function or process. As such, a KCR allows for control of the cell via light activation.

For purposes of completeness, various aspects of the present disclosure are set out in the following numbered clauses.

Aspect 1. A light gated potassium selective channelrhodopsin (KCR), comprising:
a oligomeric assembly of subunits forming a central pore, wherein each subunit comprises at least one transmembrane domain, and wherein at least one subunit comprises an amino acid substitution that alters a property of the oligomeric assembly.

Aspect 2. The light gated KCR of Aspect 1, wherein the oligomeric assembly of subunits comprises at least two subunits.

Aspect 3. The light gated KCR of Aspect 1 or 2, wherein the oligomeric assembly of subunits is selected from a dimer, a trimer, a pentamer, and a hexamer.

Aspect 4. The light gated KCR of any one of Aspects 1-3, wherein each subunit comprises seven transmembrane domains, wherein each transmembrane domain forms a helix.

Aspect 5. The light gated KCR of any one of Aspects 1-4, wherein each subunit further comprises a chromophore linked to the transmembrane domain.

Aspect 6. The light gated KCR of Aspect 5, wherein the chromophore is linked to the transmembrane domain by a Schiff base linkage.

Aspect 7. The light gated KCR of any one of Aspects 1-6, wherein the oligomeric assembly of subunits is derived from a pump like channelrhodopsin.

Aspect 8. The light gated KCR of any one of Aspects 1-7, wherein the oligomeric assembly of subunits is derived from a hyphochytrid protist.

Aspect 9. The light gated KCR of Aspect 8, wherein the hyphochytrid protist is Hyphochytrium catenoides.

Aspect 10. The light gated KCR of any one of Aspects 1-9, wherein the oligomeric assembly of subunits is derived from Hyphochytrium catenoides Kalium channelrhodopsin 1 (HcKCR1) or Hyphochytrium catenoides Kalium channelrhodopsin 2 (HcKCR2).

Aspect 11. The light gated KCR of any one of Aspects 1-10, wherein each subunit comprises an amino acid substitution that alters a property of the KCR.

Aspect 12. The light gated KCR of Aspect 11, wherein the property is selected from one or more of ion selectivity and channel kinetics.

Aspect 13. The light gated KCR of Aspect 11 or 12, wherein the property is selected from increased potassium selectivity, sodium and potassium non-selectivity, and increased τ_{off}.

Aspect 14. The light gated KCR of any one of Aspects 11-13, wherein the amino acid substitution is selected from one or more of: W102Q, D105N, Y106W, T109A, C110T, D116N, T136A, A136T, G140A, A140G, F221A, Y222A, Y222F, H225F, H225A, H225Y, and D229N.

Aspect 15. The light gated KCR of any one of Aspects 1-14, wherein the oligomeric assembly of subunits is derived from Hyphochytrium catenoides Kalium channelrhodopsin 1 (HcKCR1) or Hyphochytrium catenoides Kalium channelrhodopsin 2 (HcKCR2), and further comprises an amino acid substitution is selected from one or more of C110T, Y222A, and H225F.

Aspect 16. A nucleic acid encoding a light gated KCR of any one of Aspects 1-15.

Aspect 17. The nucleic acid of Aspect 16, further comprising a promoter.

Aspect 18. The nucleic acid of Aspect 16, wherein the promoter is selected from an inducible promoter and a constitutive promoter.

Aspect 19. The nucleic acid of any one of Aspects 16-18, wherein the coding sequence is codon optimized.

Aspect 20. A vector comprising the nucleic acid the nucleic acid of any one of Aspects 16-19.

Aspect 21. The vector of Aspect 20, wherein the vector is selected from a replication vector, a transfection vector, and an expression vector.

Aspect 22. The vector of Aspect 20 or 21, wherein the vector is selected from a plasmid, a cosmid, a virion, a viroid, a virus, and a bacterium.

Aspect 23. A method of expressing a light gated potassium selective channelrhodopsin (KCR), the method comprising:
contacting a cell with a nucleic acid of any one of Aspects 16-19 or a vector of any one of Aspects 20-22.

Aspect 24. A cell expressing the light gated KCR of any one of Aspects 1-15.

Aspect 25. The cell of Aspect 24 comprising the nucleic acid of any of one Aspects 16-19.

Aspect 26. The cell of Aspect 24 or 25, wherein the cell is a neuron.

Aspect 27. A method of optogenetically controlling a cell, the method comprising: illuminating the cell of claim 24 or 25 with a light encompassing a wavelength including the absorbance maximum of the light gated KCR of any one of Aspects 1-15, wherein illuminating the cell triggers transport of an ion into the cell.

The following examples are offered by way of illustration and not by way of limitation.

### EXPERIMENTAL

The following examples are put forth so as to provide those of ordinary skill in the art with a complete disclosure and description of how to make and use the present invention and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like

### Example 1—Methods

### Insect Cell Culture

Spodoptera frugiperda (Sf9) cells (Expression systems, authenticated by the vendor) were cultured in ESF921 medium (Expression systems) at 27.5 °C with 130 rpm in an InnovaS44i R shaking incubator (Eppendorf).

### HEK293 Cell Culture

HEK293FT cells (Thermo Fisher, authenticated by the vendor) were maintained in a 5% CO₂ humid incubator with DMEM media (GIBCO) supplemented with 10% FBS (Invitrogen), and 1% Penicillin-Streptomycin (Invitrogen), and were enzymatically passaged at 90% confluence by trypsinization.

### Cloning, Protein Expression, and Purification of HcKCR1 and HcKCR2

Wild-type HcKCR1 (M1-S265) was modified to include an N-terminal influenza hemagglutinin (HA) signal sequence and FLAG-tag epitope, and C-terminal enhanced green fluorescent protein (eGFP), followed by 10× histidine and Rho1D4 epitope tags; the N-terminal and C-terminal tags are removable by human rhinovirus 3C protease cleavage. Wild type HcKCR2 (M1-D265) was modified to include C-terminal Kir2.1 membrane targeting sequence, human rhinovirus 3C protease cleavage sequence, enhanced green fluorescent protein (eGFP), and 8× histidine tag.

The constructs were expressed in Spodoptera frugiperda (Sf9) insect cells using the pFastBac baculovirus system. Sf9 insect cells were grown in suspension to a density of 3.0×10⁶ cells ml⁻¹, infected with baculovirus and shaken at 27.5 °C for 24 h. All-trans retinal (ATR) (Sigma-Aldrich) is supplemented to a final concentration of 10 μM in the culture medium 24 h after the infection. The cell pellets were lysed with a hypotonic lysis buffer (20 mM HEPES-NaOH pH 7.5, 20 mM NaCl, 10 mM MgCl₂, 1 mM benzamidine, 1 g ml⁻¹ leupeptin, 10 μM ATR), and cell pellets were collected by centrifugation at 10,000×g for 30 min. The above process was repeated twice; then, cell pellets were disrupted by homogenizing with a glass dounce homogenizer in a hypertonic lysis buffer (20 mM HEPES-NaOH pH 7.5, 1 μM NaCl, 10 mM MgCl₂, 1 mM benzamidine, 1 g ml⁻¹ leupeptin, 10 μM ATR), and crude membrane fraction was collected by ultracentrifugation (45Ti rotor, 125,000×g for 1 h). The above process was repeated twice; then, the membrane fraction was homogenized with a glass douncer in a membrane storage buffer (20 mM HEPES-NaOH pH 7.5, 500 mM NaCl, 10 mM imidazole, 20% glycerol, 1 mM benzamidine, 1 kg ml⁻¹ leupeptin), flash frozen in liquid nitrogen, and stored at −80 °C until use.

The membrane fraction was solubilized in a solubilization buffer (1% n-dodecyl-β-D-maltoside (DDM) (EMD Millipore), 20 mM HEPES-NaOH pH 7.5, 500 mM NaCl, 20% glycerol, 5 mM imidazole, 1 mM benzamidine, 1 kg ml⁻¹ leupeptin) and solubilized at 4 °C for 2 h. The insoluble cell debris was removed by ultracentrifugation (45Ti rotor, 125,000×g, 1 h), and the supernatant was mixed with the Ni-NTA superflow resin (QIAGEN) at 4 °C for 2 h. The Ni-NTA resin was loaded onto an open chromatography column, washed with 2.5 column volumes of wash buffer (0.05% DDM, 20 mM HEPES-NaOH pH7.5, 100 mM NaCl, and 25 mM imidazole) three times, and eluted by elution buffer (0.05% DDM, 20 mM HEPES-NaOH pH7.5, 100 mM NaCl, and 300 mM imidazole). After tag cleavage by His-tagged 3C protease, the sample was reapplied onto the Ni-NTA open column to trap the cleaved eGFP-His-tag and His-tagged 3C protease. The flow-through fraction was collected and concentrated to approximately 2 mg ml⁻¹ using an Amicon ultra 50 kDa molecular weight cutoff centrifugal filter unit (Merck Millipore). The concentrated samples were ultracentrifuged (TLA 55 rotor, 71,680×g for 30 minutes) before size-exclusion chromatography on a Superdex 200 Increase 10/300 GL column (Cytiva), equilibrated in DDM SEC buffer (0.03% DDM, 20 mM HEPES-NaOH pH7.5, 100 mM NaCl). The peak fractions of the protein were collected and concentrated to approximately 10 mg ml⁻¹.

### Preparation of Membrane Scaffold Protein

Membrane scaffold protein (MSP1D1E3) is expressed and purified as described earlier with the following modifications. Briefly, MSP1D1E3 gene in pET-43a(+) was transformed in Escherichia coli (E. coli) BL21 (DE3) cells. Cells were grown at 37 °C with shaking to an OD600 of 0.5-1.0, and then expression of MSP1D1E3 was induced by addition of 1 mM IPTG. Cells were further grown for at 37 °C 4 hr, and cells were harvested by centrifugation. Cell pellets were resuspended in PBS (−) buffer supplemented with 1% Triton X-100 and protease inhibitors and were lysed by sonication. The lysate was centrifuged at 30,000×g for 30 min, and the supernatant was loaded onto a Ni-NTA column equilibrated with lysis buffer, followed by washing with four bed volumes of wash buffer-1 (40 mM HEPES-NaOH pH7.5, 300 mM NaCl, 1% Triton X-100), four bed volumes of wash buffer-2 (40 mM HEPES-NaOH pH7.5, 300 mM NaCl, 50 mM sodium cholate), four bed volumes of wash buffer-3 (40 mM HEPES-NaOH pH7.5, 300 mM NaCl), four bed volumes of wash buffer-4 (40 mM HEPES-NaOH pH7.5, 300 mM NaCl, 20 mM imidazole), and eluted with wash buffer-4 containing 300 mM imidazole. The eluted MSP1D1E3 was dialyzed in buffer containing 10 mM HEPES-NaOH pH7.5, 100 mM NaCl, and concentrated to approximately 10 mg ml-1 using an Amicon ultra 10 kDa molecular weight cutoff centrifugal filter unit (Merck Millipore). The concentrated samples were ultracentrifuged (TLA 55 rotor, 71,680×g for 30 minutes), and stored at −80 °C after flash freezing in liquid nitrogen. The concentration was determined by absorbance at 280 nm (extinction coefficient=29,910 M⁻¹ cm⁻¹) measured by NanoDrop 2000c spectrophotometer (Thermo scientific).

### Nanodisc Reconstitution of HcKCR1 and HcKCR2

Prior to nanodisc reconstitution, 30 mg SoyPC (Sigma P3644-25G) was dissolved in 500 μL chloroform and dried using a nitrogen stream to form a lipid film. The residual chloroform was further removed by overnight vacuum desiccation. Lipid films were rehydrated in buffer containing 1% DDM, 20 mM HEPES-NaOH pH7.5, 100 mM NaCl, resulting in a clear 10 mM lipid stock solution.

HcKCR1 was reconstituted into nanodiscs formed by the scaffold protein MSP1E3D1 and SoyPC at a molar ratio of 1:4:400 (monomer ratio: HcKCR, MSP1E3D1, SoyPC). First, freshly purified HcKCR1 in SEC buffer (0.05% DDM, 20 mM HEPES-NaOH pH7.5, 100 mM NaCl) was mixed with SoyPC and incubated on ice for 20 min. Purified MSP1D1E3 was then added to mess up to total solution volume of 750 μL, and gently mixed on rotator at 4 °C for 10 min. Final concentrations were 14.5 μM HcKCR1, 58.2 μM MSP1E3D1, and 5.8 mM SoyPC, respectively. Detergents were removed by stepwise addition of Bio-Beads SM2 (Bio-Rad). Prior to use, Bio-Beads were washed by sonication in methanol, water, and buffer containing 20 mM HEPES-NaOH pH7.5, 100 mM NaCl with an ultrasonic bath sonicator and weighed after liquid was removed by a P1000 tip. As the first batch, 100 mg Bio-Beads (final concentration of 133 mg ml⁻¹) was added, and the mixture was gently rotated at 4 °C for 12 h. The second batch of Bio-Beads (equal amount) was added and further rotated at 4 °C for 2.5 h. The Bio-Beads were removed by passage through a PolyPrep column (Bio-Spin column, Bio-Rad), and the lysate was ultracentrifuged (TLA 55 rotor, 71,680×g for 30 minutes) before size-exclusion chromatography on a Superdex 200 Increase 10/300 GL column (Cytiva), equilibrated in buffer containing 20 mM HEPES-NaOH pH7.5, 100 mM NaCl. The peak fractions were collected and concentrated to approximately 6 mg ml⁻¹ estimated based on the absorbance (A 280) value of 16, using an Amicon ultra 50 kDa molecular weight cutoff centrifugal filter unit (Merck Millipore).

HcKCR2 was reconstituted into nanodiscs basically in the same manner as HcKCR1. In brief, HcKCR2, MSP1D1E3 and SoyPC were mixed at a molar ratio of 1:4:400, with the final concentration of 41 μM, 164 μM, and 4.1 mM, respectively. The total solution volume was 750 μL. Detergents were removed by stepwise addition of Bio-Beads SM2 (Bio-Rad). The first Bio-Beads batch amount was 25 mg. After rotation at 4 °C for 12 h, 40 mg of fresh Bio-Beads were added every 12 h, twice in total. HcKCR2 in a nanodisc was purified through size-exclusion chromatography and concentrated to approximately 12 mg ml⁻¹ estimated based on the absorbance (A 280) value of 30, using an Amicon ultra 50 kDa molecular weight cutoff centrifugal filter unit (Merck Millipore).

### Cryo-EM Grid Preparation of Nanodisc-Reconstituted HcKCR1 and HcKCR2

Prior to grid preparation, the sample was centrifuged at 20,380×g for 30 min at 4 °C. The grids were glow-discharged with a PIB-10 plasma ion bombarder (Vacuum Device) at 10 mA current with the dial setting of 2 min for both side. 3 μL of protein solution was applied to freshly glow-discharged Quantifoil R1.2/1.3 Au 300 mesh holey carbon grid in dark room with dim red light. Samples were vitrified by plunging into liquid ethane cooled by liquid nitrogen with a FEI Vitrobot Mark IV (Thermo Fisher Scientific) at 4 °C with 100% humidity. The blotting force was set as 10. The waiting and blotting time were 10 s and 4 s, respectively.

### Cryo-EM Data Acquisition and Image Processing of HcKCR1

Cryo-EM images were acquired at 300 kV on a Krios G3i microscope (Thermo Fisher Scientific) equipped with a Gatan BioQuantum energy filter and a K3 direct detection camera in the electron counting mode. The movie dataset was collected in standard mode, using a nine-hole image shift strategy in the SerialEM software, with a nominal defocus range of −0.8 to −1.6 m. The 5,445 movies were acquired at a dose rate of 14.313 e⁻/pixel/s, at a pixel size of 0.83 Å and a total dose of 48 e⁻/Å².

The data processing was performed using the cryoSPARC v3.2.0 software packages. The collected 5,445 movies were subjected to patch motion correction and patch CTF refinement in cryoSPARC. Initial particles were picked from all micrographs using blob picker and were extracted using a box size of 280 pixels. 407,781 particles were selected after 2D classification from 2,439,182 particles. The following ab initio reconstruction, heterogeneous refinement, and non-uniform refinement enable reconstruction of a 2.92 Å map (C1 symmetry) with 130,130 particles. Further particles were picked by template picker and Topaz picker and subjected to 2D classification followed by heterogeneous refinement. Non-uniform refinement after removing of the duplicated particles enable obtention of a 2.60 Å map (C3 symmetry) with 917,464 particles. The following 2D classification, global CTF refinement, and non-uniform refinement yielded the final map at a global resolution of 2.58 Λ.

Cryo-EM data Acquisition and Image Processing of HcKCR2

Cryo-EM images were acquired at 300 kV on a Krios G4 microscope (Thermo Fisher Scientific) equipped with a Gatan BioQuantum energy filter and a K3 direct detection camera in the electron counting mode. The movie dataset was collected in standard mode, using the fringe free imaging (FFI) and aberration-free image shift (AFIS) strategy in the EPU software (Thermo Fisher Scientific), with a nominal defocus range of −0.6 to −1.6 μm. The 7,718 movies were acquired at a dose rate of 17.5 e⁻/pixel/s, at a pixel size of 0.83 Å and a total dose of 51 e⁻/Å².

The data processing was performed using the cryoSPARC v3.3.2 software packages. The collected 7,718 movies were subjected to patch motion correction and patch CTF refinement in cryoSPARC. Particles were picked from all micrographs by blob picker, template picker, and Topaz picker, resulted in 3,382,955 particles, 5,852,598 particles, and 2,844,575 particles, respectively. These particle subsets were subjected to 2D classification and subsequent heterogeneous refinement. The particles in the best classes were 508,364 particles for blob picker, 777,572 particles for template picker, and 519,445 particles for Topaz picker, respectively. After removal of duplicates, 1,243,623 particles were selected and subjected to non-uniform refinement, resulting in a 2.66 Å map. The additional heterogeneous refinement, non-uniform refinement, local motion correction, and another non-uniform refinement along with defocus refinement and global CTF refinement yielded the final map at a global resolution of 2.53 Å.

### Model Building and Refinement

An initial model of HcKCR1 was formed by rigid body fitting of the predicted models of HcKCR1, generated using locally installed AlphaFold2. This starting model was then subjected to iterative rounds of manual and automated refinement in Coot and Refmac5 in Servalcat pipeline, respectively. The Refmac5 refinement was performed with the constraint of C3 symmetry. The initial model for HcKCR2 was the refined model of HcKCR1.

The final model was visually inspected for general fit to the map, and geometry was further evaluated using Molprobity. The final refinement statistics is summarized in Table S1. All molecular graphics figures were prepared with UCSF Chimera, UCSF ChimeraX, CueMol2 and PyMOL).

### Pore Analysis

The ion-conducting pore pathways were calculated by the software HOLLOW 1.3 with a grid-spacing of 1.0 Å.

### Measurement of UV Absorption Spectra and pH Titration

To investigate the pH dependence of the absorption spectrum of HcKCR1 and HcKCR2, 10 mg ml⁻¹ purified protein solution was 100-fold diluted in buffer containing 0.05% DDM, 100 mM NaCl, and 100 mM of either citric acid pH2.2, citric acid pH 3.0, sodium acetate pH 4.0, sodium citrate pH 5.0, sodium cacodylate pH 6.0, HEPES-NaOH pH7.0, Tris-HCl pH8.0, N-cyclohexyl-2-aminoethanesulfonic acid (CHES) pH 9.0, 3-(cyclohexylamino)-1-propanesulfonic acid (CAPS) pH 10.0, or CAPS pH 11.0. The StockOptions pH Buffer Kit (Hampton research) was used for buffer preparation except for CHES pH 9.0 (Nacalai). The absorption spectra were measured with a V-750 UV-visible spectrometer (JASCO) at room temperature.

### Laser Flash Photolysis

For the laser flash photolysis spectroscopy, HcKCR1 wildtype and D105N were reconstituted in azolectin (11145, Sigma-Aldrich, Merck, Germany) with a protein-to-lipid molar ratio of 1:50 in 100 mM KCl, 20 mM HEPES-KOH pH 7.5. OD of the proteo-liposome suspensions was adjusted to ~0.8 (protein concentration ~0.2-0.3 mg/mL) at the absorption maximum wavelengths. The laser flash photolysis measurement was conducted as previously described. Nano-second pulses from an optical parametric oscillator (5.7 mJ/pulse cm², basiScan, Spectra-Physics, CA) pumped by the third harmonics of Nd-YAG laser (λ=355 nm, IND140, Spectra-Physics, CA) were used for the excitation of HcKCR1 wildtype and D105N at λ_{exc}=510 and 500 nm, respectively. The transient absorption spectra were obtained by monitoring the intensity change of white-light from a Xe-arc lamp (L9289-01, Hamamatsu Photonics, Japan) passed through the sample with an ICCD linear array detector (C8808-01, Hamamatsu, Japan). To increase the signal-to-noise (S/N) ratio, 45-60 spectra were averaged, and the singular-value-decomposition (SVD) analysis was applied. To measure the time-evolution of transient absorption change at specific wavelengths, the output of a Xe-arc lamp (L9289-01, Hamamatsu Photonics, Japan) was monochromated by monochromators (S-10, SOMA OPTICS, Japan) and the change in the intensity after the photo-excitation was monitored with a photomultiplier tube (R10699, Hamamatsu Photonics, Japan). To increase the S/N ratio, 100-200 signals were averaged.

To measure the transient absorption change of pyranine due to proton release and uptake by HcKCR1 wildtype, the protein was solubilized in 100 mM KCl, 0.05% DDM, and pH was adjusted to 7.2 close to the pKa of pyranine by adding NaOH, and then 40 μM pyranine (L11252, Wako, Japan) was added. The formation and disappearance of the protonated form of pyranine were monitored at 454 nm by subtracting the transient absorption change obtained without pyranine from that obtained with pyranine as previously reported.

### High Performance Liquid Chromatography (HPLC) Analysis of Retinal Isomers in HcKCR1

The HPLC analysis of retinal isomers was conducted as described elsewhere with a slight modification. The purified sample was incubated at 4 °C overnight in the dark prior to the HPLC analysis. A 30-μL sample and 120 μL of 90% (v/v) methanol aqueous solution and 10 μL of 2 M hydroxylamine (NH₂OH) were added to the sample. Then, retinal oxime hydrolyzed from the retinal chromophore in HcKCR1 was extracted with 500 μL of n-hexane. A 200 μL of the extract was injected into an HPLC system equipped with a silica column (particle size 3 μm, 150×6.0 mm; Pack SIL, YMC, Japan), a pump (PU-4580, JASCO, Japan), and a UV-visible detector (UV-4570, JASCO, Japan). As the mobile-phase solvent, n-hexane containing 15% ethyl acetate and 0.15% ethanol was used at a flow rate of 1.0 mL min⁻¹. Illumination was performed with green light (510±5 nm) for 60 s. The molar composition of the retinal isomers the sample was calculated with the molar extinction coefficient at 360 nm for each isomer (all-trans-15-syn: 54,900 M⁻¹ cm⁻¹; all-trans-15-anti: 51,600 M⁻¹ cm⁻¹; 13-cis-15-syn, 49,000 M⁻¹ cm⁻¹; 13-cis-15-anti: 52,100 M⁻¹ cm⁻¹; 11-cis-15-syn: 35,000 M⁻¹ cm⁻¹; 11-cis-15-anti: 29,600 M⁻¹ cm⁻¹; 9-cis-15-syn: 39,300 M⁻¹ cm⁻¹; 9-cis-15-anti: 30,600 M⁻¹ cm⁻¹).

### Laser Patch Clamp

The electrophysiological assays of HcKCR1 were carried out using ND7/23 cells, as described previously with a slight modification. Briefly, ND7/23 cells were grown in Dulbecco's modified Eagle's medium (D-MEM, FUJIFILM Wako Pure Chemical Co., Osaka, Japan) supplemented with 5% fetal bovine serum (FBS) under a 5% CO₂ atmosphere at 37 °C. Eight hours after the transfection, the medium was replaced by D-MEM containing 5% FBS, 50 ng/mL nerve growth factor-7S (Sigma-Aldrich, St. Louis, MO), 1 mM N6,2′-O-dibutyryladenosine-3′,5′-cyclic monophosphate sodium salt (Nacalai tesque, Kyoto, Japan), and 1 μM Cytosine-1-β-D(+)-arabinofuranoside (FUJIFILM Wako Pure Chemical Co., Osaka, Japan). The coding sequence of HcKCR1 was fused to a Kir2.1 membrane trafficking signal, eYFP, and an ER-export signal. The gene was cloned into a vector behind a CMV-promotor and the expression plasmids were transiently transfected in ND7/23 cells using Lipofectamine™ 3000 transfection reagent (Thermo Fisher Scientific Inc., Waltham, MA) and electrophysiological recordings were conducted at 2-3 days after the transfection. The transfected cells were identified by the presence of eYFP fluorescence under an up-right microscope (BX50WI, Olympus, Tokyo, Japan).

All experiments were carried out at room temperature (20-22 °C). Currents were recorded using an EPC-8 amplifier (HEKA Electronic, Lambrecht, Germany) under a whole-cell patch clamp configuration. The internal pipette solution contained 121.2 mM KOH, 90.9 mM glutamate, 5 mM Na₂EGTA, 49.2 mM HEPES, 2.53 mM MgCl₂, 2.5 mM MgATP, 0.0025 mM ATR (pH 7.4 adjusted with HCl). Extracellular solution contained 138 mM NaCl, 3 mM KCl, 2.5 mM CaCl₂, 1 mM MgCl₂, 4 mM NaOH, and 10 mM HEPES at pH 7.4 (with 11 mM glucose added up to 310 mOsm). The pipette resistance was adjusted to 3-6 MΩ (3.7±0.4, n=7) with a series resistance of 6-11 MΩ (8.3±0.8) and a cell capacitance of 32-216 pF (83±21) with the extracellular/intracellular solutions. In every experiment, the series resistance was compensated.

While voltage-clamping at a holding potential, a laser flash (3-5 ns) at 532 nm (Nd:YAG laser, Minilite II, Continuum, San Jose, CA) was illuminated through an objective lens (LUMPlan FL 40×, NA 0.80 W, Olympus, Japan). The timing of laser flash was set to be time 0 according to the photodiode response under the sample. The measurements were conducted with a holding potential of 0 mV at every 15 s. The data were filtered at 1 kHz, sampled at 250 kHz (Digidata1440 A/D, Molecular Devices Co., Sunnyvale, CA), collected using pClamp10.3 software (Molecular Devices Co., Sunnyvale, CA), and stored in a computer. Five current responses were averaged and served for the following analyses. Using the simplex method of nonlinear least-squares (IgorPro 9, WaveMetrics, Portland, OR), the kinetics of photocurrent were fitted by a triple-exponential function.

### ATR-FTIR Spectroscopy

Ion binding to HcKCR1 was monitored by ATR-FTIR spectroscopy as described previously, except for some minor modifications for reconstitution into the membrane. In ATR-FTIR spectroscopy, rhodopsins are normally reconstituted into lipids to form a film on the ATR-prism. Thus, sample was reconstituted with a protein-to-lipid (asolectin; Sigma-Aldrich) molar ratio of 1:20, by removing the n-dodecyl-β-D-maltoside (DDM) with Bio-Beads (SM-2, Bio-Rad) at 4 °C in dark condition. The HcKCR1 sample in asolectin liposomes was washed repeatedly with a buffer containing 2 mM K₂HPO₄/KH₂ PO₄ (pH 7.5) and collected by ultracentrifuging for 20 min at 222,000×g at 4 °C in dark condition. The lipid-reconstituted HcKCR1 was placed on the surface of a silicon ATR crystal (Smiths, three internal total reflections) and naturally dried. The sample was then rehydrated with the buffer at a flow rate of 0.6 ml mir-, and temperature was maintained at 20 °C by circulating water. The perfusion buffer is composed of 200 mM NaCl, 200 mM Tris-HCl, pH 7.5 (buffer A) and 200 mM KCl, 200 mM Tris-HCl, pH 7.5 (buffer B). In the case of anion binding experiments, the perfusion buffer was replaced with 200 mM NaCl, 20 mM HEPES-NaOH, pH 7.5 (buffer A) and 200 mM NaBr, 20 mM HEPES-NaOH, pH 7.5 (buffer B), respectively.

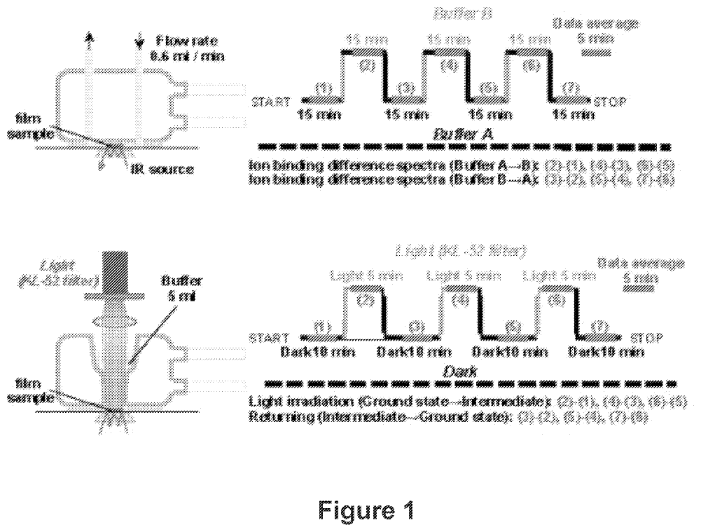

ATR-FTIR spectra were recorded in kinetics mode at 2 cm⁻¹ resolution, renge of 4000-700 cm⁻¹ using an FTIR spectrometer (Agilent) equipped with a liquid nitrogen-cooled mercury-cadmium-telluride (MCT) detector (an average of 1710 interferograms per 15 min). Ion binding-induced difference spectra were measured by exchanging the buffer A and buffer B. The cycling procedure is shown in FIG. 1, and the difference spectra were calculated as the averaged spectra in buffer B minus buffer A. The spectral contributions of the unbound salt, the protein-lipid swelling/shrinkage, and the water-buffer components were corrected as described previously.

Light-induced structural changes of HcKCR1 were also measured by ATR-FTIR as shown in FIG. 1. Since ATR-FTIR experimental setup has been optimized for ion perfusion-induced difference spectroscopy using a solution exchange system, we have modified experimental setup that enables light irradiation experiment. A light source was installed above the ATR prism. In addition, an optical filter and a condenser lens were placed directly under the light source. To obtain the ion binding-induced difference spectra under the light illumination condition, light minus dark difference spectra under perfusing the different solution between buffer A and buffer B was subtracted from each other. The spectral contributions of the unbound salt, the protein-lipid swelling/shrinkage, and the water-buffer components were also corrected as described previously.

### In Vitro Electrophysiology

Cells and devices for the measurement were prepared as described. Briefly, HEK293 cells (Thermo Fisher) expressing opsins were placed in an extracellular tyrode medium (150 mM NaCl, 4 mM KCl, 2 mM CaCl₂, 2 mM MgCl₂, 10 mM HEPES pH 7.4, and 10 mM glucose). Borosilicate pipettes (Harvard Apparatus, with resistance of 4-6 MOhm) were filled with intracellular medium (140 mM potassium-gluconate, 10 mM EGTA, 2 mM MgCl₂ and 10 mM HEPES pH 7.2). Light was delivered with the Lumencor Spectra X Light engine with 470 nm and 560 nm filters for blue and orange light delivery, respectively.

Channel kinetics and photocurrent amplitudes were measured in voltage clamp mode at 0 mV (before liquid junction potential correction) holding potential and then analyzed in Clampfit software (Axon Instruments) after smoothening using a lowpass Gaussian filter with a −3 dB cutoff for signal attenuation and noise reduction at 1,000 Hz. Liquid junction potentials were corrected using the Clampex built-in liquid junction potential calculator as previously described (Kishi et al., 2022). Equilibrium potentials were measured by holding membrane potentials from −96 mV (after LJP correction) in steps of 10 mV.

Statistical analysis was performed with one-way ANOVA and the Kruskal-Wallis test for non-parametric data, using Prism 7 (GraphPad) software. Data collection across opsins was randomized and distributed to minimize across-group differences in expression time, room temperature, and related experimental factors.

### Ion Selectivity Testing in HEK293 Cells

HEK293 cells and devices for the measurement were prepared as described in the previous section. For the high sodium extracellular/high potassium intracellular condition, a sodium bath solution containing 150 mM NaCl, 2 mM CaCl₂, 2 mM MgCl₂, 10 mM HEPES pH 7.3 with 10 mM glucose was used, along with potassium pipette solution containing 150 mM KCl, 2 mM CaCl₂, 2 mM MgCl₂, 10 mM HEPES pH 7.2, and 10 mM glucose. For the high potassium extracellular/high sodium intracellular condition, NaCl and KCl concentrations were reversed, and all other ionic concentrations were kept constant. Liquid junction potentials were corrected using the Clampex built-in liquid junction potential calculator.

For sodium extracellular/potassium intracellular condition, equilibrium potentials were measured by holding membrane potentials from −124 mV to +16 mV in steps of 10 mV (after UP correction). for potassium extracellular/sodium intracellular, equilibrium potentials were measured by holding from −26 mV to 104 mV (after LJP correction) in 10 mV steps. The relative ion permeability of sodium and potassium (P_{K}/P_{Na}) was calculated using the Goldman-Hodgkin-Katz equation, with T=298K, P_{CL}=0, F=96485 C/mol and R=8.314 J/K-mol.

$${V}_{m} = \frac{RT}{F} ln ( \frac{{{p}_{K} [ {K}^{+} ]}_{o} + {{p}_{Na} [ {Na}^{+} ]}_{o} + {{p}_{Cl} [ {Cl}^{-} ]}_{o}}{{{p}_{K} [ {K}^{+} ]}_{i} + {{p}_{Na} [ {Na}^{+} ]}_{i} + {{p}_{Cl} [ {Cl}^{-} ]}_{i}} )$$

To test intracellular Guanidinium blockage, an intracellular buffer containing 150 mM GuHCl, 2 mM CaCl₂, 2 mM MgCl₂, 10 mM HEPES pH 7.3, and 10 mM glucose was used with a regular high sodium extracellular buffer.

Statistical analysis was performed with one-way ANOVA and the Kruskal-Wallis test for non-parametric data, using Prism 7 (GraphPad) software. Data collection across opsins was randomized and distributed to minimize across-group differences in expression time, room temperature, and related experimental factors.

### System Setup for Molecular Dynamics Simulations

Simulations of HcKCR1 WT and a HcKCR1 D116N mutant were performed. The simulations were initiated using structure reported in this manuscript. For the D1 16N mutant simulations, the mutation was introduced while maintaining the positions of all the common atoms. To study the exit pathway of ions from the channel, a K⁺ was placed in both the intracellular and extracellular vestibules near D166 and D205, respectively. For each simulation condition, three independent simulations were performed, each 500 ns in length. For each simulation, initial atom velocities were assigned randomly and independently.

The structure was aligned to the Orientations of Proteins in Membranes (Lomize et al., 2006) entry for 1M0L(Schobert et al., 2002) (bacteriorhodopsin). Prime (Schrödinger)(Jacobson et al., 2002) was used to model missing side chains, and to add capping groups to protein chain termini. The Crosslink Proteins tool (Schrödinger) was used to model unresolved portions of ECL2, ICL3, and ECL3. Parameters for the ligands were generated using the Paramchem webserver (Vanommeslaeghe and MacKerell, 2012; Vanommeslaeghe et al., 2010, 2012). Dowser software was used to add waters to cavities within the protein structure(Zhang and Hermans, 1996). Six POPC lipids were modeled in the center of the trimer; three in the extracellular leaflet and three in the intracellular leaflet. Protonation states of all titratable residues were assigned at pH 7. Histidine residues were modeled as neutral, with a hydrogen atom bound to either the delta or epsilon nitrogen depending on which tautomeric state optimized the local hydrogen-bonding network. Using Dabble(Betz, 2017), the prepared protein structures were inserted into a pre-equilibrated palmitoyl-oleoyl-phosphatidylcholine (POPC) bilayer, the system was solvated, and potassium and chloride ions were added to neutralize the system and to obtain a final concentration of 150 mM. The final systems comprised approximately 101,000 atoms, and system dimensions were approximately 105×105×95 Å.

### Molecular Dynamics Simulation and Analysis Protocols

The CHARMM36m force field was used for proteins; the CHARMM36 force field was used for lipids and ions; and the TIP3P model was used for waters. Retinal parameters were obtained through personal communication with Scott Feller. All simulations were performed using the Compute Unified Device Architecture (CUDA) version of particle-mesh Ewald molecular dynamics (PMEMD) in AMBER18 on graphics processing units (GPUs).

Systems were first minimized using three rounds of minimization, each consisting of 500 cycles of steepest descent followed by 500 cycles of conjugate gradient optimization. 10.0 and 5.0 kcal·mol⁻¹·Å⁻² harmonic restraints were applied to protein, lipids, and ligand for the first and second rounds of minimization, respectively. 1 kcal·mol⁻¹·Å⁻² harmonic restraints were applied to protein and ligand for the third round of minimization. Systems were then heated from 0 K to 100 K in the NVT ensemble over 12.5 ps and then from 100 K to 298 K in the NPT ensemble over 125 ps, using 10.0 kcal·mol⁻¹·Å⁻² harmonic restraints applied to protein and ligand heavy atoms. Subsequently, systems were equilibrated at 298 K and 1 bar in the NPT ensemble, with harmonic restraints on the protein and ligand non-hydrogen atoms tapered off by 1.0 kcal·mol⁻¹·Å⁻² starting at 5.0 kcal·mol⁻¹·Å⁻² in a stepwise fashion every 2 ns for 10 ns, and then by 0.1 kcal·mol⁻¹·Å⁻² every 2 ns for 20 ns. Production simulations were performed without restraints at 310 K and 1 bar in the NPT ensemble using the Langevin thermostat and the Monte Carlo barostat, and using a timestep of 4.0 fs with hydrogen mass repartitioning(. Bond lengths were constrained using the SHAKE algorithm. Non-bonded interactions were cut off at 9.0 Å, and long-range electrostatic interactions were calculated using the particle-mesh Ewald (PME) method with an Ewald coefficient of approximately 0.31 Å, and 4th order B-splines. The PME grid size was chosen such that the width of a grid cell was approximately 1 Å. Trajectory frames were saved every 200 ps during the production simulations.

### Quantification and Statistical Analysis

Statistical analysis was performed with one-way ANOVA and the Kruskal-Wallis test for non-parametric data, using Prism 7 (GraphPad) software. Data collection across opsins was randomized and distributed to minimize across-group differences in expression time, room temperature, and related experimental factors.

### Example 2—Overall Structural Comparison Between HcKCR1, HcKCR2, and ChRmine

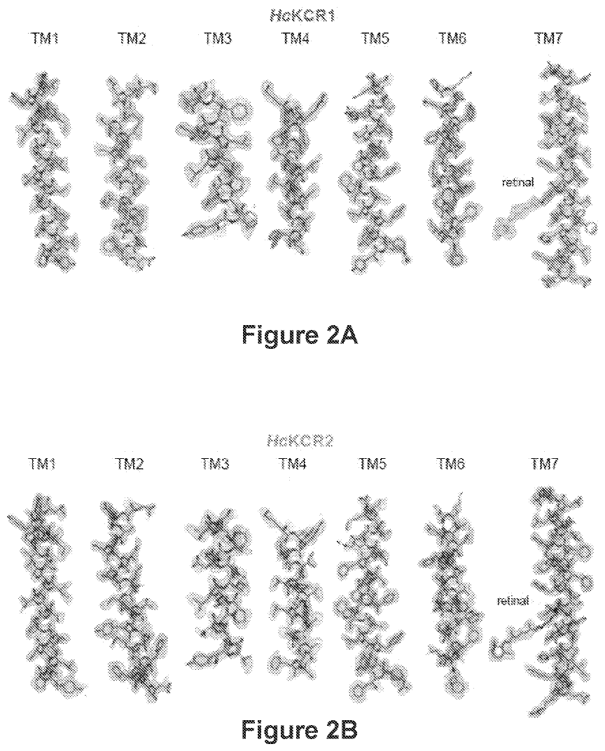

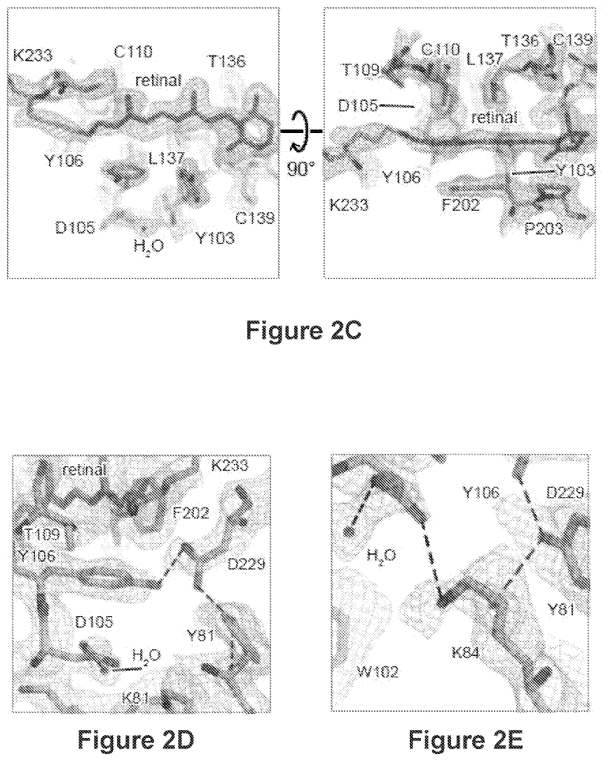

For structural determination, HcKCR1 and 2 (residues 1-265 for both) were expressed in Sf9 insect cells and reconstituted the purified proteins into lipid nanodiscs formed by the scaffold protein MSP1E3D1 and soybean lipids (STAR methods). Using cryo-EM, the structures of the HcKCR1 and 2 were solved in the dark state to overall resolutions of 2.6 Å and 2.5 Å, respectively. The high-resolution density maps allowed for accurate modelling of the vast majority of both HcKCRs (residues 6-260 for HcKCR1 and 2-260 for HcKCR2), as well as water molecules, lipids, and the all-trans retinal whose conformer was also validated by high-performance liquid chromatography (HPLC) analysis (FIGS. 2A-2I, 3A, and 3B). The N-terminal residue of HcKCR2, P2, is surrounded by four residues (P95, F96, W100, and Y101) in the structure and there is no space for the first methionine (FIG. 2H). This is consistent with previous findings revealing that the first methionine is post-translationally cleaved off when the second and third residues are proline and non-proline, respectively (the third residue is phenylalanine in HcKCR2).

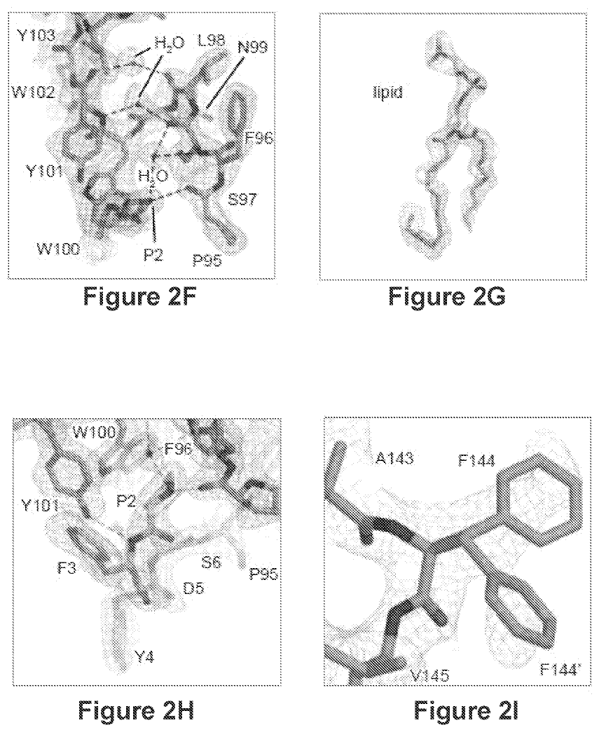

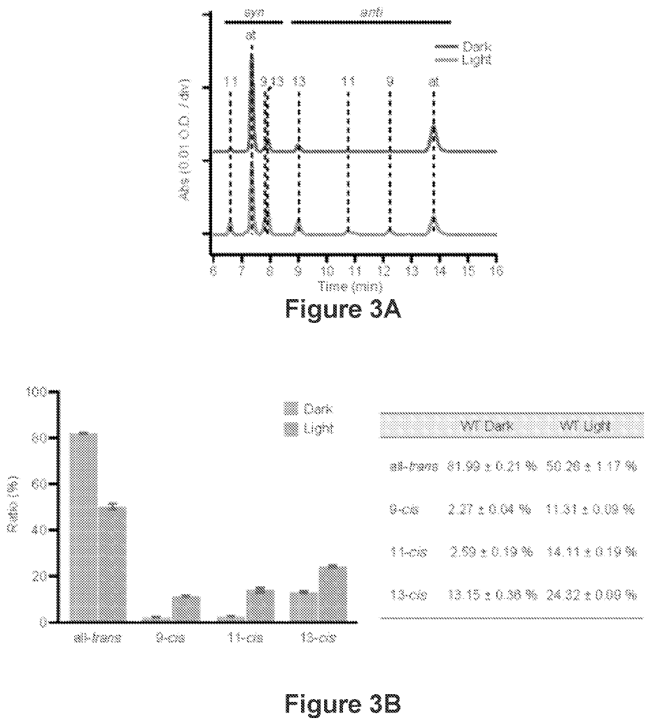

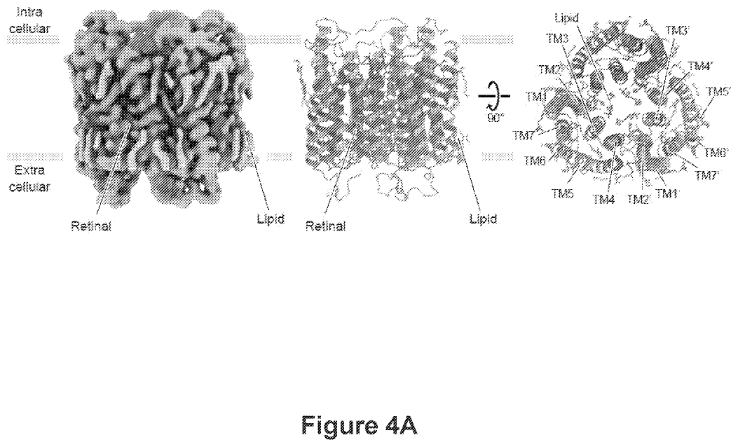

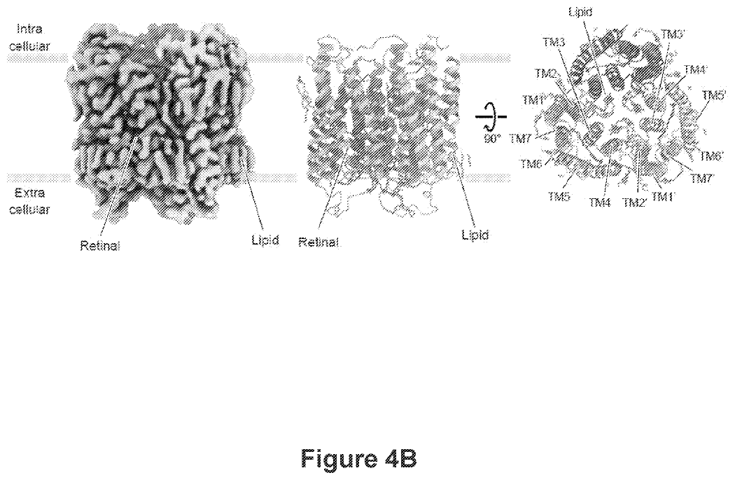

Both HcKCR1 and 2 form a trimer (FIGS. 4A and 4B), as was also observed in ChRmine, the only PLCR for which high-resolution structural information is available. The trimerization is mainly achieved by the direct and lipid-mediated interactions among transmembrane helices (TMs) 1-2 and TMs 4-5 of adjacent protomer, and the center of the trimer interface is filled with six lipid molecules (FIGS. 4A and 4B). The monomer of HcKCR1 and 2 consists of an extracellular N-terminal region (residues 6-21 for HcKCR1 and 2-21 for HcKCR2), an intracellular C-terminal region (residues 255-260 for both), and 7-TM domains (within residues 22-254 for both), connected by three intracellular loops (ICL1-3) and three extracellular loops (ECL1-3) (FIGS. 4C and 4D). The overall structures of HcKCR1 and 2 are almost identical with a Ca root-mean-square deviation (r.m.s.d.) of only 0.51 Å and only minor differences in the N-terminal region, ICLs, and ECLs (FIG. 4E).

HcKCRs also superpose well onto ChRmine, but with several structural differences (Ca r.m.s.d. between HcKCR1 and ChRmine is 1.75 Å) (FIG. 4F). First, both the N- and C-terminal regions in ChRmine have short α-helices running almost parallel to the membrane, which are absent in the HcKCRs (FIG. 4F). Second, except for ECL3, all ICLs and ECLs have significantly different conformations. ECL1 in particular, which distinguishes PLCRs from the rest of the ChR families, is ~6 residues shorter than ChRmine, and the entire loop is packed more closely to the core of the helix bundle (FIG. 4F). Third, TM1 and the C-terminal half of TM7 are tilted about 7 and 10 degrees, respectively, relative to the rest of the helical bundle. The C-terminal TM7 helix is also ~1.5 turns longer than that of ChRmine (FIG. 4F), making it more similar to that of canonical CCRs such as C1C2 (the chimera derived from CrChR1 and CrChR2) (FIG. 4G). In PLCRs, residues from TM1, 2, 3, 7, and ECL1 form the core of the ion-conducting pathway within each monomer, so the structural differences of TM1, 7, and ECL1 observed in HcKCRs change the shape of the pathway, to be discussed in more details later.

### Example 3—The Schiff Base Region

Microbial rhodopsins have an all-trans retinal molecule covalently bound to a conserved lysine residue on TM7 via a Schiff base linkage. The Schiff base is protonated in the dark and this positive charge must be stabilized by one or more nearby acidic residues for efficient isomerization of retinal. Initial reactions triggered by light absorption include retinal isomerization and subsequent proton transfer from the Schiff base to a nearby acidic residue or water molecule. The residues stabilizing the Schiff base proton and receiving the proton in the photo-intermediate state (M intermediate) have been historically termed the Schiff base counterion(s) and the proton acceptor, respectively. The precise architecture of the Schiff base region is closely linked to several key properties of microbial rhodopsins, so we next focused on this region.

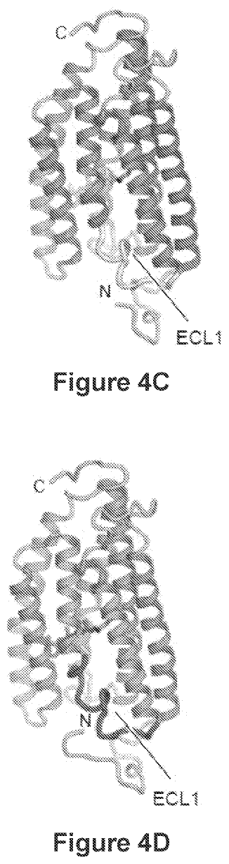

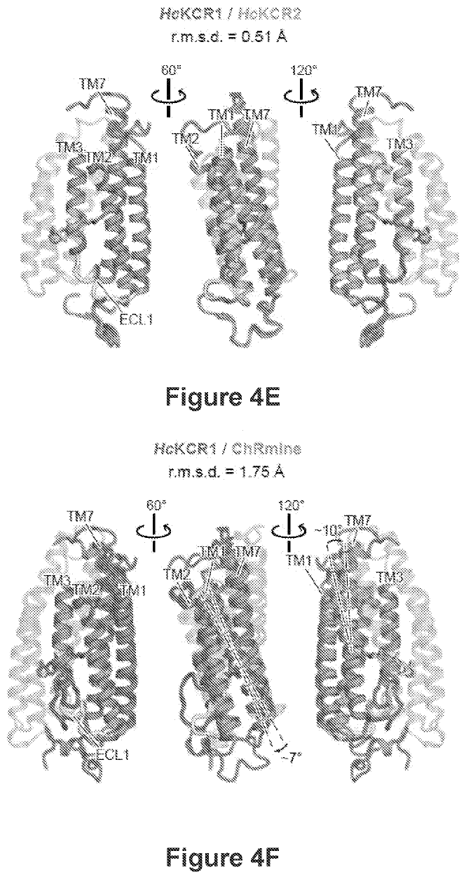

A previous study revealed that the Schiff base region of ChRmine is strikingly different from those of other microbial rhodopsins; TM3 is unwound in the middle of the membrane, and two aspartates, the strong candidates for the Schiff base counterion and proton acceptor, are placed on TM3/ECL1 and TM7. The first aspartate (D115 in ChRmine) faces away from the Schiff base proton, and the second aspartate (D253 in ChRmine) is fixed by two hydrogen bonds with Y85 on TM2 and Y116 on TM3 (FIG. 5A, right). These features were also observed in HcKCR1 and 2, suggesting that the architecture of the Schiff base region is conserved among PLCRs (FIG. 5A). However, there are still several differences between HcKCR1, 2, and ChRmine: K84 points towards the extracellular side in HcKCRs (FIGS. 5A and 2E); no water molecules are observed between the Schiff base proton and the two aspartates (D105 and D229 in HcKCRs) in HcKCRs (FIG. 5A); and the highly conserved arginine residue on ECL1 (R112 in ChRmine) is replaced by a tryptophan residue (W102 in HcKCRs) in HcKCRs (FIG. 5A). These differences motivated us to further characterize the functions of D105 and D229.

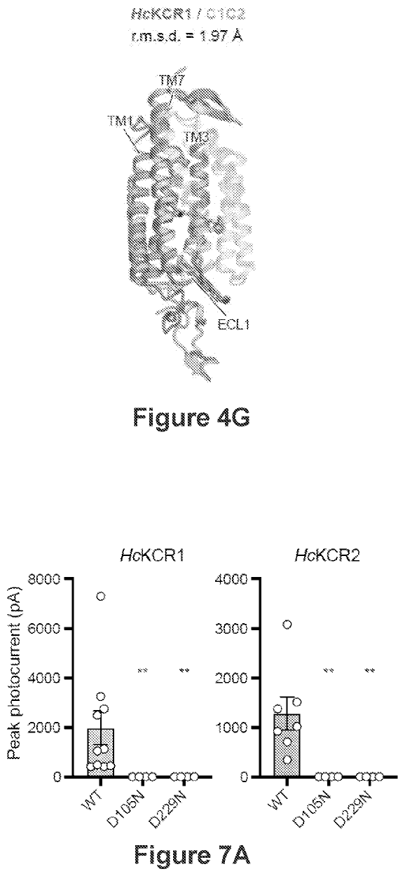

First, to assign protonation states of these two aspartates and to identify which aspartate works as the primary counterion, we measured the absorption spectra of wild-type (WT), D105N, and D229N mutants of both HcKCRs (FIGS. 5B, 6A, and 6B). The λₘₐₓ of WT, D105N, and D229N mutants of HcKCR1 at neutral pH is 521, 508, and 386 nm, respectively, demonstrating that protonation of D105 causes only a small blue-shift in the absorption spectrum (~13 nm), while protonation of D229 causes a much larger blue-shift (~135 nm), which can be explained by concomitant deprotonation of the Schiff base nitrogen (FIG. 5B, left). The same trend was also observed in HcKCR2 (FIG. 5B, right). This finding suggests that both D105 and D229 are deprotonated in the dark state, but only the deprotonation of D229 is necessary to stabilize the positive charge of the Schiff base proton; in other words, D229 is the primary counterion. This is strikingly different from ChRmine, in which both of the corresponding aspartate residues (D115 and D253) are essential counterions of the Schiff base proton.

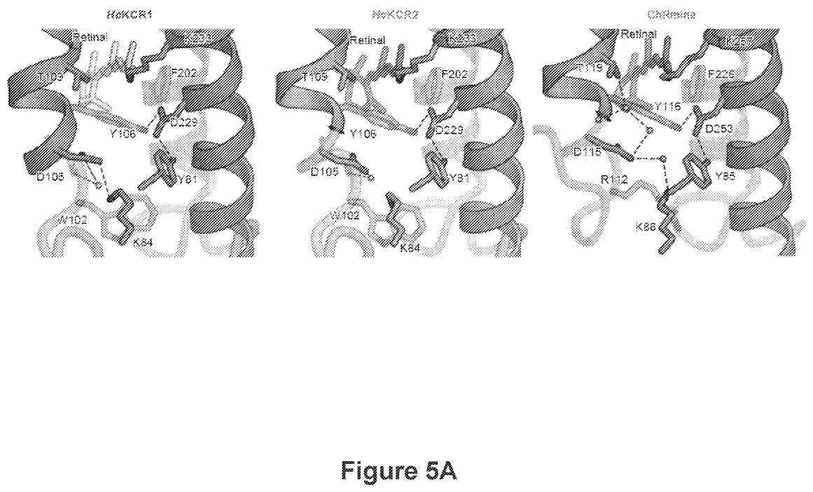

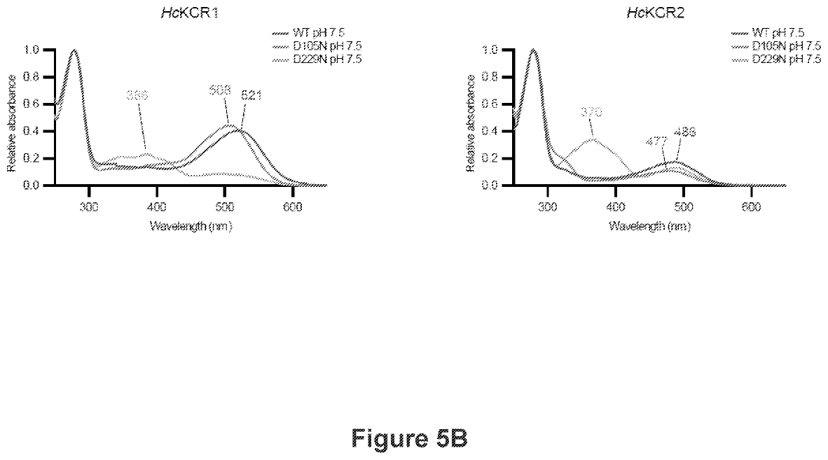

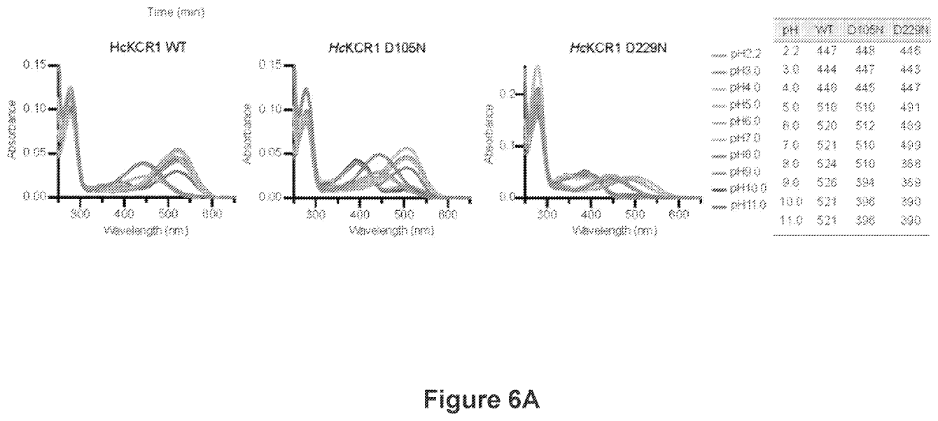

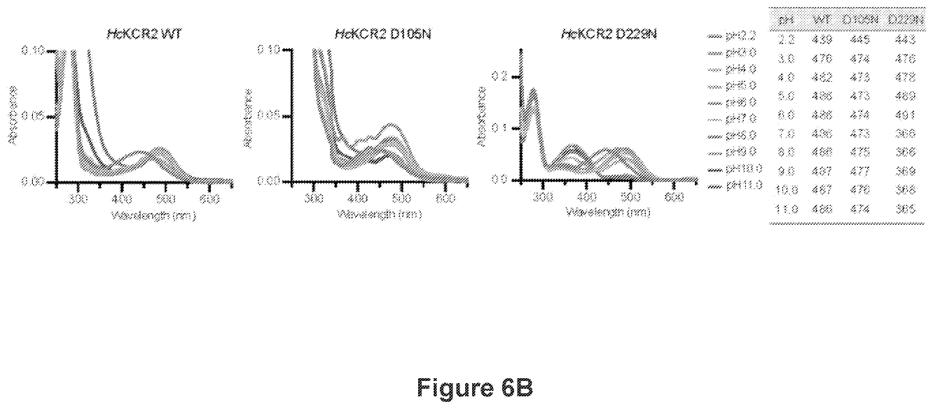

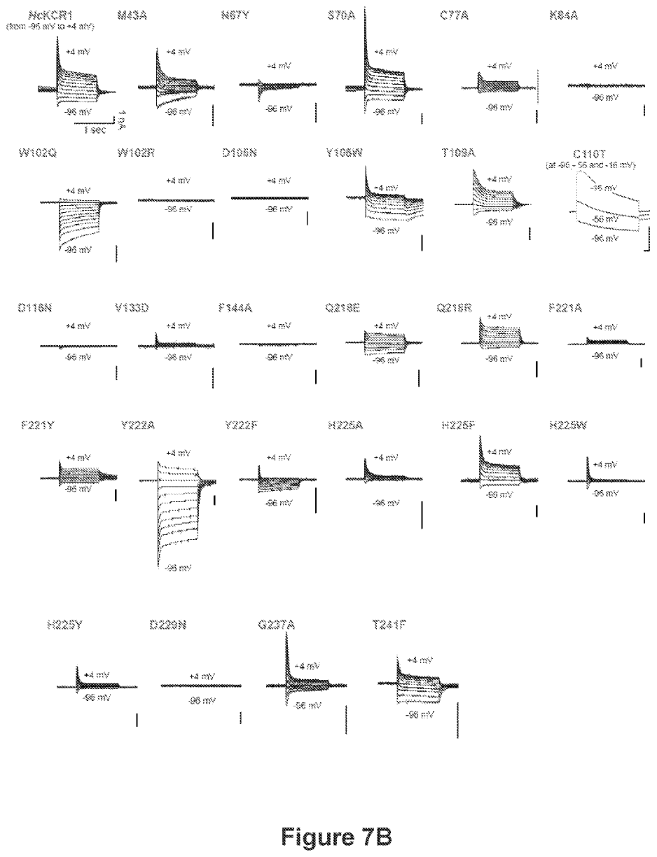

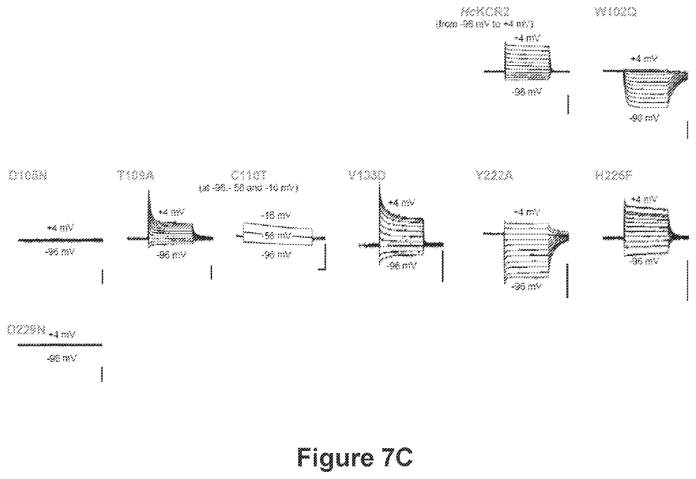

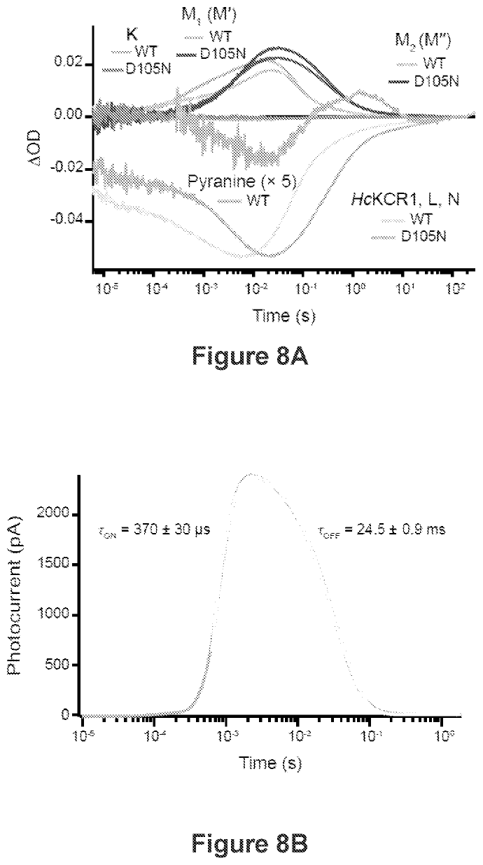

However, surprisingly, the electrophysiology experiments with these mutants showed that channel function is completely abolished not only for D229N but also for D105N (FIGS. 7A-7C). To understand the reason, we next performed laser flash photolysis and laser patch clamp experiments (FIGS. 8A-8E). These experiments revealed that HcKCR1 has eight intermediates (K₁, K₂, L₁, L₂, M₁, M₂, N₁, N₂) in its photocycle, with M₁ and M₂ representing the open state, consistent with a previous study. We further measured the photocycle of the D105N mutant and found that the rise and decay of M intermediate become significantly slower in this mutant (FIGS. 8A and 8C), suggesting that D105 works as the proton acceptor. This interpretation is also supported by the flash photolysis experiment of WT HcKCR1 in the presence of pyranine, a pH-sensitive dye, showing that the Schiff base proton is released to the bulk solvent later than the rise of the M₁ (FIG. 8A); this result indicates that the Schiff base proton is not directly released to water but is transferred to an acidic residue in the Schiff base region. Notably, the shapes of the absorption spectra of M₁ and M₂ intermediates in the D105N mutant are significantly different from those in WT HcKCR1 (FIG. 8E), thus it can be inferred that the structures of D105N mutant in these intermediates, which are newly denoted as M′ and M″ (FIG. 8E), are also different from those of WT, and thereby channel function of this mutant is compromised. Overall, these results suggest that D105 does not work as the Schiff base counterion in the dark state but works as the proton acceptor in the M intermediate, and the proton transfer to the D105 would be an important step for correct channel gating.

### Example 4—The Retinal Binding Pocket

The residues surrounding the retinal chromophore are important determinants of key ChR properties, including kinetics and absorption spectrum.

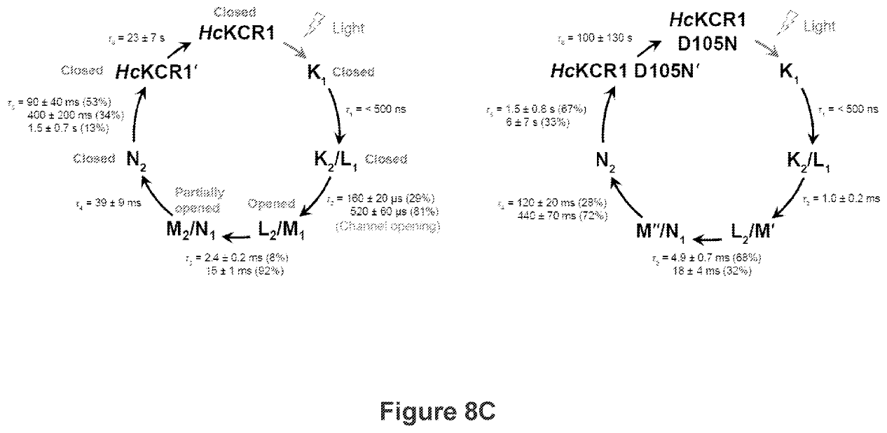

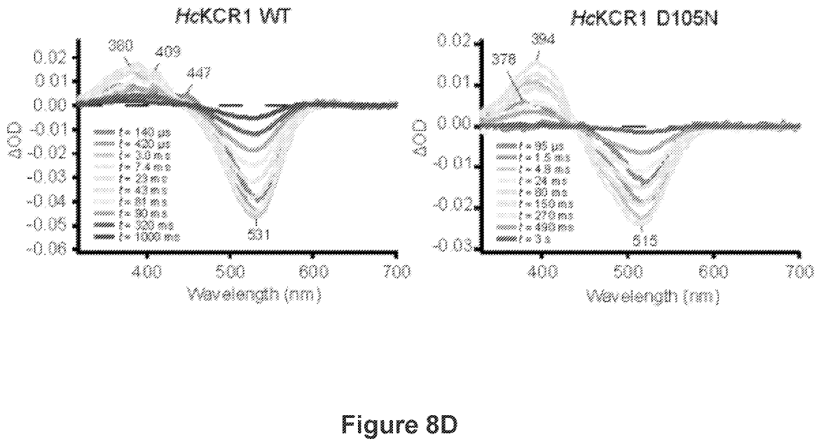

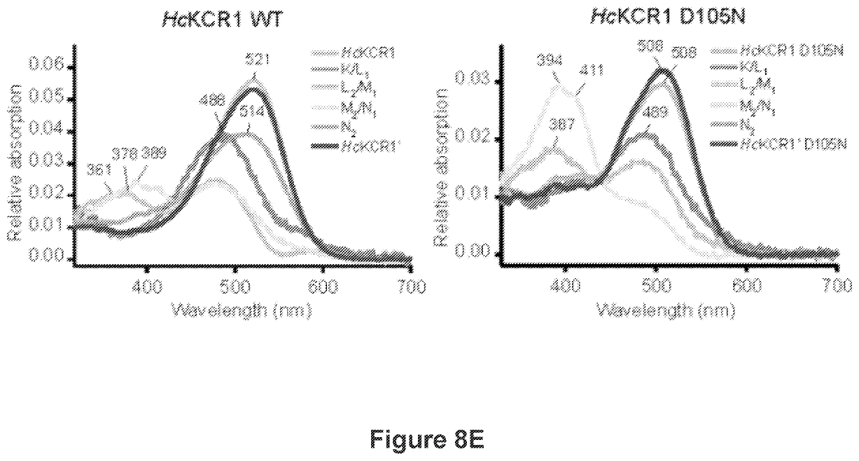

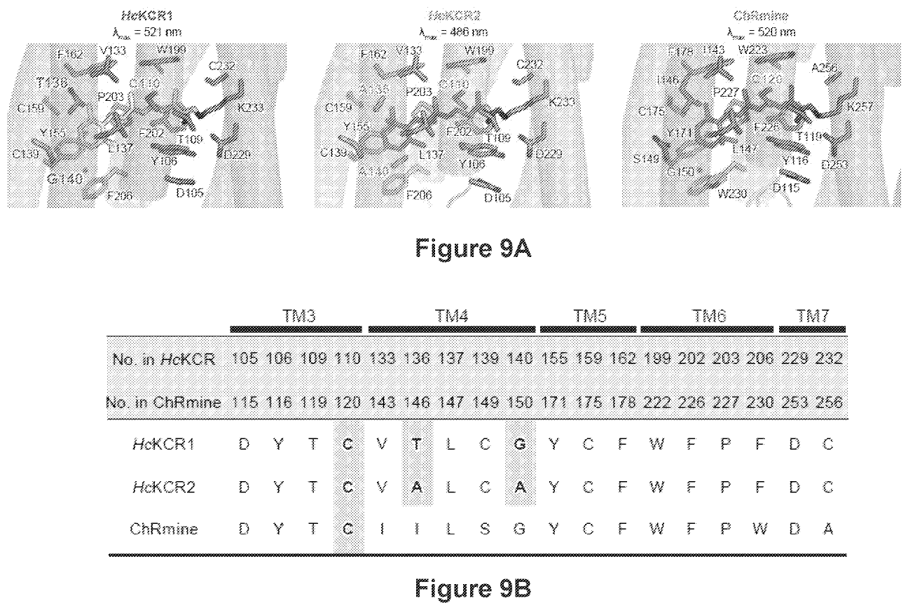

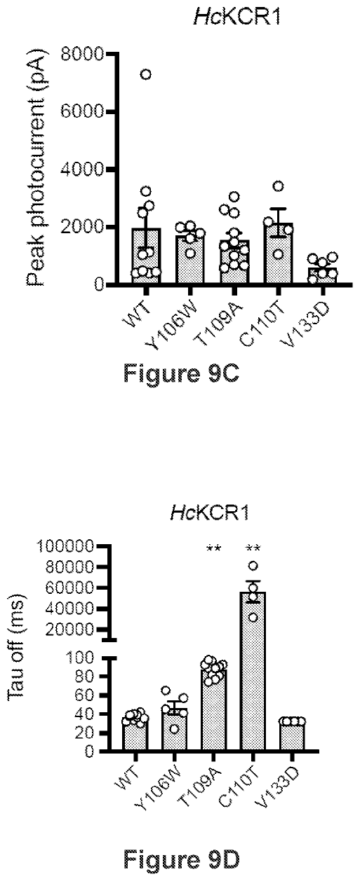

The residues comprising the retinal binding pocket are very similar between HcKCR1, 2, and ChRmine (FIG. 9A); 12 of 18 residues are conserved between HcKCRs and ChRmine, and only two residues are different between HcKCR1 and 2 (FIG. 9B). To understand the function of these residues, mutations to Y106 and T109 in HcKCRs (Y116 and T119 in ChRmine) were introduced because a previous study showed that Y116W and T119A mutations in ChRmine significantly decelerate and accelerate off kinetics, respectively. However, it was found that the effects of corresponding mutations in HcKCRs are very different; the Y106W mutation moderately decelerates the off kinetics of only HcKCR2, and the T109A mutation does not accelerate but decelerates the off kinetics of only HcKCR1 (FIGS. 9C, 9D, 7B, 9E, and 9F). Y106 and T109 are part of the retinal binding pocket as well as part of the Schiff base region (FIG. 5A), thus the differences in the Schiff base region described above likely account for the differences in mutational effects among HcKCR1, 2, and ChRmine.

Next, mutations were introduced to C110 and V133, for which threonine, serine, or alanine mutants are known to significantly prolong off-kinetics in CrChR2, giving rise to the step-function opsins (SFOs) which have found broad optogenetic application in neuroscience. Although a previous attempt to transfer this mutation to PLCRs was not successful, the result was strikingly successful here for both HcKCR1 and 2 (FIGS. 9C and 9D). The C110T mutation increased the τ_{off} of HcKCR1 and 2 by ~1500- and ~1800-fold, respectively (FIG. 9D); notably, the HcKCR1 C110T mutant still shows comparable channel activity to WT (FIG. 9C). As far it is known, this is the first study to create a step-function opsin in the PLCR family, and the HcKCR1 C110T mutant is expected to work as a powerful optogenetic tool for long-timescale inhibition.

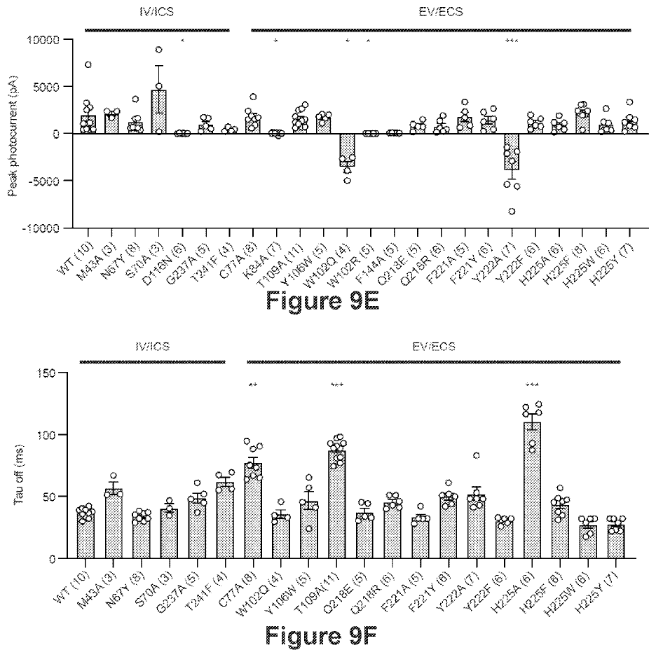

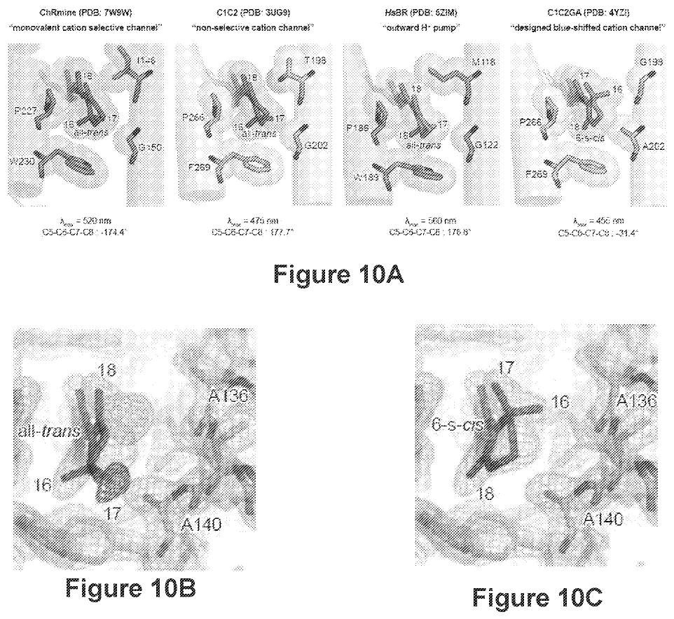

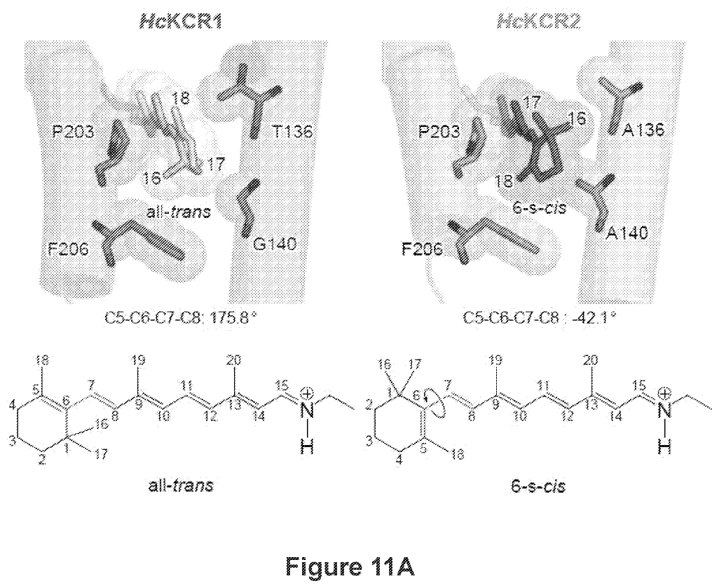

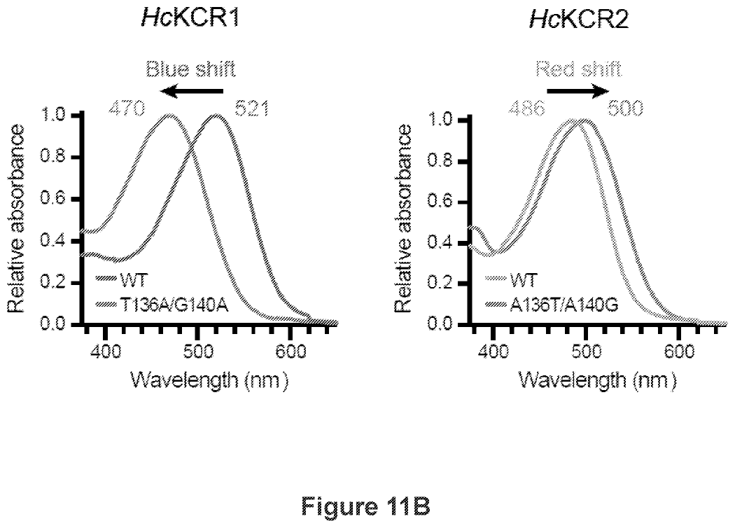

HcKCR1 and 2 show different spectral properties; λₘₐₓ of HcKCR1 and 2 is 521 nm and 486 nm, respectively (FIG. 5B). The retinal binding pockets of HcKCR1 and 2 are very similar, with the only differences at positions 136 and 140 near the β-ionone ring of the retinal (FIGS. 9A and 9B), providing an excellent opportunity to test spectral mechanisms. In all reported structures of naturally-occurring microbial rhodopsins, the retinal has a 6-s-trans form in the binding pocket (FIG. 10A). However, during the structural refinement of HcKCR2 (STAR Methods), it was noticed that the 6-s-trans retinal exhibits a significant steric clash between the C₁₇ atom of the retinal and the methyl group of A140, and strong extra density was observed next to the C₁₃ atom (FIG. 10B). This suggests that the β-ionone ring should be rotated in the HcKCR2 structure, and surprisingly, when 6-s-cis retinal was modeled, this conformation perfectly fits the density (FIG. 10C). This result indicates that these two residues (A136 and A140 in HcKCR2) create a steric clash with the C₁₇ atom and simultaneously make space to accommodate the C₁₆ atom, to induce the rotation of the β-ionone ring (FIG. 11). The ~220 degrees rotation of the ring shrinks the π-conjugated system of retinal and thereby induces a ~35 nm spectral shift (FIGS. 5B and 11A). This is in good agreement with a previous study that showed a designed ChR with glycine and alanine at the same positions, C1C2GA, exhibits retinal rotated by ~210 degrees and a spectrum blue-shifted by ~20 nm (FIG. 10A). To further test this hypothesis, these two residues were swapped between HcKCR1 and 2 and confirmed that the T136A/G140A mutation to HcKCR1 and A136T/A140G mutation to HcKCR2 cause the predicted blue- and red-shifts, respectively (FIG. 11B). The impact of these two residues determining the orientation of the β-ionone ring in the binding pocket largely explains the spectral difference between HcKCR1 and 2. To present knowledge, HcKCR2 is the first naturally-occurring microbial rhodopsin for which a 6-s-cis configuration of the retinal has been experimentally demonstrated.

### Example 5—Ion-conducting Pore and K⁺ Selectivity

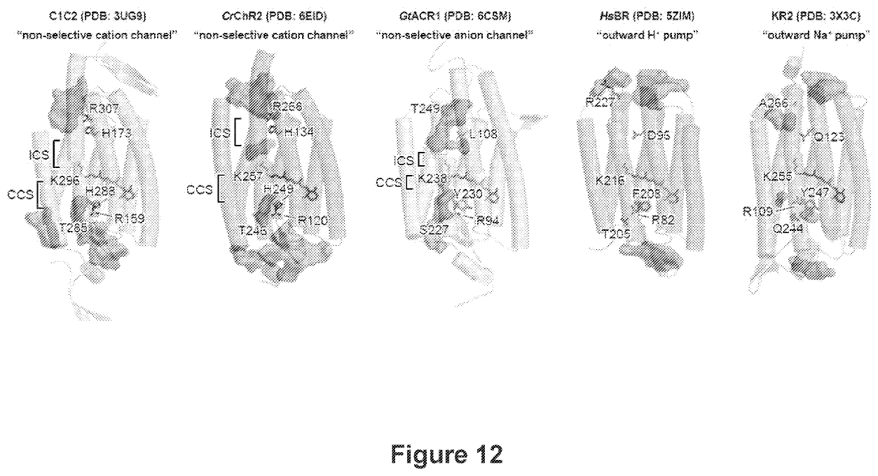

The three major classes of ChRs including the PLCRs, although assembling as multimers, each possess an ion-conducting pore within the monomer, formed by TM1, 2, 3, and 7. For example, the PLCR ChRmine was discovered to form a trimer with a large opening in the middle of the trimer; although mutations in this region can modulate ion selectivity, this opening was not predicted or shown to form a conducting pore for ChRmine in a previous study. In the dark state, the monomer pore is divided into the intracellular and extracellular vestibules (IV and EV) by two or three constriction sites, which are called intracellular, central, and extracellular constriction sites (ICS, CCS, and ECS) (FIG. 12).

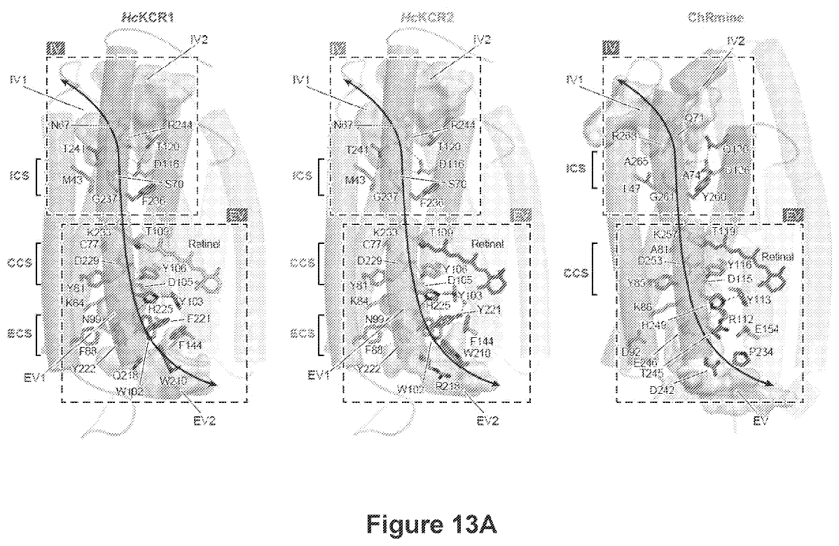

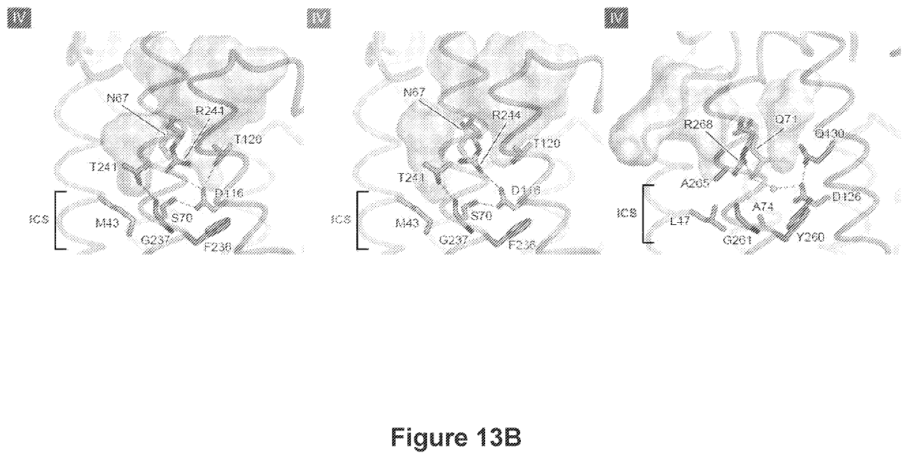

HcKCRs have a relatively similar sequence to archaeal pump-type rhodopsins, but with larger cavities due to structural differences of the pore-forming helices (FIGS. 13A and 12). Notably, due to the unwinding of TM3 in the middle of the membrane, not only TM1, 2, 3, and 7, but also ECL1, significantly contribute to the creation of the EV, as observed in the ChRmine structure (FIGS. 4F and 13A). The overall location of the cavities in HcKCRs is very similar to ChRmine but three notable differences are observed between them.

First, both HcKCRs and ChRmine exhibit two IVs (IV1 and IV2) divided by a conserved arginine on TM7 (R244 in HcKCRs and R268 in ChRmine), and they are occluded by the ICS, but the interaction network in ICS is significantly different. In ChRmine, R268 forms the H-bond with Q71, and D126 has direct H-bond interactions with both Q130 and Y260 and water-mediated H-bond interaction with Q71. This H-bond network, together with L47, A74, and G261, makes the ICS (FIG. 4B, right). In contrast, in HcKCRs, R244 approaches TM3 because of the ~10 degrees tilt of the cytoplasmic half of TM7 (FIG. 1F) and forms a salt bridge with D116 (D126 in ChRmine). Moreover, A74, Q130, and Y260 in ChRmine are replaced by S70, T120, and F236, respectively, resulting in the significant rearrangement of the H-bond network centered on D116 (FIG. 13B, left and middle).

Second, the EV in HcKCRs extends deeper into the core of the bundle than ChRmine and indeed reaches the Schiff base (FIG. 13C). While the architecture of the Schiff base region is similar between HcKCRs and ChRmine (FIG. 5A), the small conformational difference of K84 enlarges the pore in HcKCRs, and the EV extends close to the Schiff base-forming lysine (K233), as observed in GtACR1 (FIG. 12). As a result, not only the counterion complexes (D155, D229, Y81, and Y106) but also C77, T109, and K233 contribute to defining the CCS in HcKCRs (FIG. 13C, left and middle).

Finally, and most importantly, the shape and the surface property of HcKCRs' EV are strikingly different from that of ChRmine (FIG. 13C, left and middle). Several hydrophilic residues that line the EV in ChRmine, including D92, R112, E154, T245, and E246 are replaced by aromatic residues in HcKCRs (F88, W102, F144, F/Y221, and Y222), and they make the EV's surface more hydrophobic (FIG. 13C, left and middle). Moreover, W102 and Y222 protrude to the center of EV and make a new constriction (ECS) with ECL1. As described earlier, HcKCRs' ECL1 is positioned closer to the core of the helix bundle compared to that of ChRmine (FIG. 4F) and allows N99 on ECL1 to forms a H-bond with Y222 and thereby separate the EV into two cavities (FIG. 13C, left and middle). The replacement of arginine with tryptophan (W102) in HcKCRs also causes the rotameric change of histidine (H225 in HcKCRs) and generates a new H-bond between H225 and F/Y221 (FIG. 13C, left and middle). Overall, these aromatic residues create unique EVs whose shape and properties are different from other microbial rhodopsins (FIG. 12).

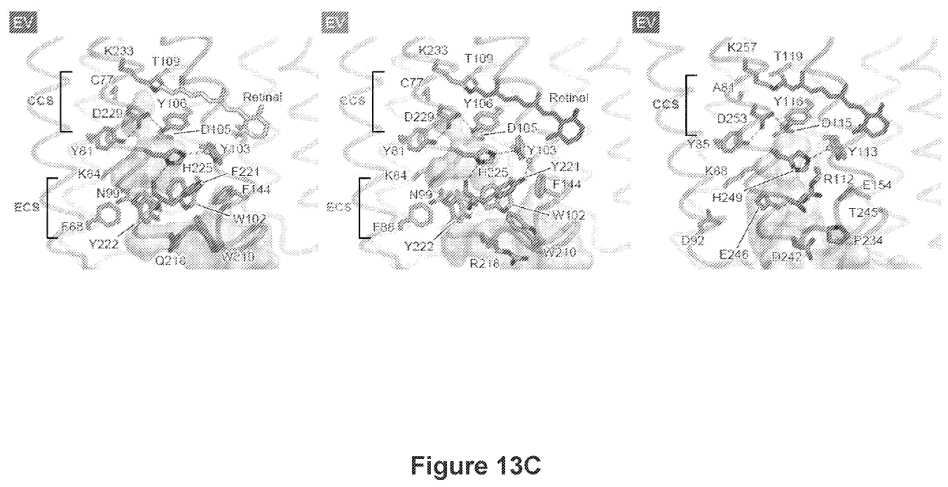

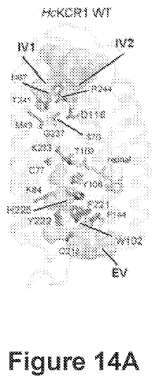

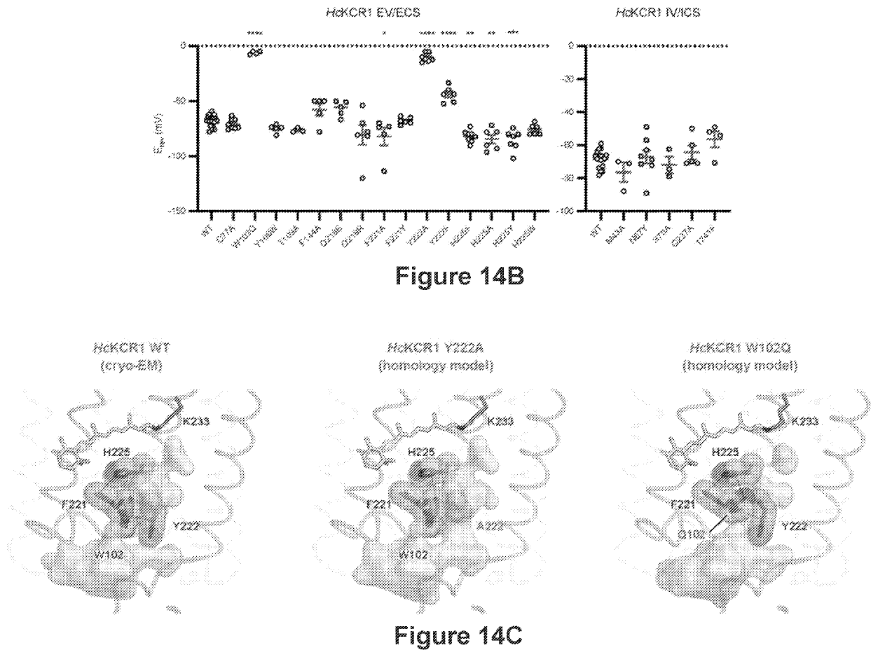

To understand the mechanism of K⁺ selectivity by KCRs, mutations were introduced to the residues placed along the IVs and EVs of HcKCR1 and measured their reversal potentials (Eᵣₑᵥ) (FIGS. 14A, 14B, 7B, 9E, and 9F). WT HcKCR1 exhibits Eᵣₑᵥ of −68.4±1.3 mV and a permeability ratio (P_{K}/P_{Na}) of 25.7±2.7, consistent with a previous study, indicating function as a K⁺-selective channel with minor Na⁺ conductance. It was found that most mutations had negligible effects on selectivity, but the mutations to W102, D116, F221, Y222, and H225 caused significant changes in Eᵣₑᵥ (FIGS. 14A, 14B, 7B, 9E, and 9F). Strikingly, it was found that mutants F221A, H225F, H225A, and H225Y became more selective to K⁺, with significantly hyperpolarized Eᵣₑᵥ (−82.1±8.1, −82.0±1.9, −84.6±4.0, and −85.4±3.5 mV, respectively) (FIG. 14B). By contrast, W102Q and Y222A mutants became almost non-selective to Na⁺ vs. K⁺ with strikingly depolarized Eᵣₑᵥ (−6.25±1.5 and −10.6±1.5 mV, respectively). A more conservative mutation at the Y222 position (Y222F) caused an intermediate depolarization in Eᵣₑᵥ (−44.2±2.9 mV), suggesting that K⁺ selectivity tracks with bulkiness of the side chains comprising the EV. This finding also agrees well with the previously reported data indicating that KCR selectivity is inversely proportional to the size of hydrated substrate cations. These four bulky aromatic residues are localized in the EV, suggesting that W102, F221, Y222, and H225 could assemble and form an ion selectivity filter in HcKCR1 (FIG. 14A). This idea is further supported by the homology models of Y222A and W102Q mutants created from the WT cryo-EM structure; the packing interactions between these four residues become weaker in both mutants, and especially Y222A mutant shows a strikingly enlarged cavity compared to WT (FIGS. 14C and 14D). The disruption of the constriction at the filter region would cause the loss of K⁺ selectivity in these mutants.

Do these residues select for K⁺? The EV, where these residues are located, is actually the exit site for the substrate K⁺ under typical ion balance conditions, as under physiological electrochemical gradients, K⁺ flows preferentially from the intracellular to the extracellular side (while Na⁺ flows in the opposite direction). Since the EV will predominantly serve as the exit site for K⁺ as well as the entry site for Na⁺ under physiological conditions, it was considered that HcKCR1 might achieve K⁺ selectivity under typical chemical gradients (high K⁺/low Na⁺ intracellularly and low K⁺/high Na⁺ extracellularly) chiefly by preventing entry of Na⁺ from the extracellular side via these aromatic amino acids. With robust outward K⁺ currents flowing, little inward Na⁺ would be expected to flow (especially if an aromatic size-exclusion filter deterred flux of the larger hydrated Na⁺ ion in the presence of high flux of the smaller hydrated K⁺ ion). But inward Na⁺ currents would still be possible in the absence of competing K⁺ ions from outside the cell, if at strongly negative membrane potentials such that intracellular K⁺ ions were no longer flowing outward down an electrochemical gradient.

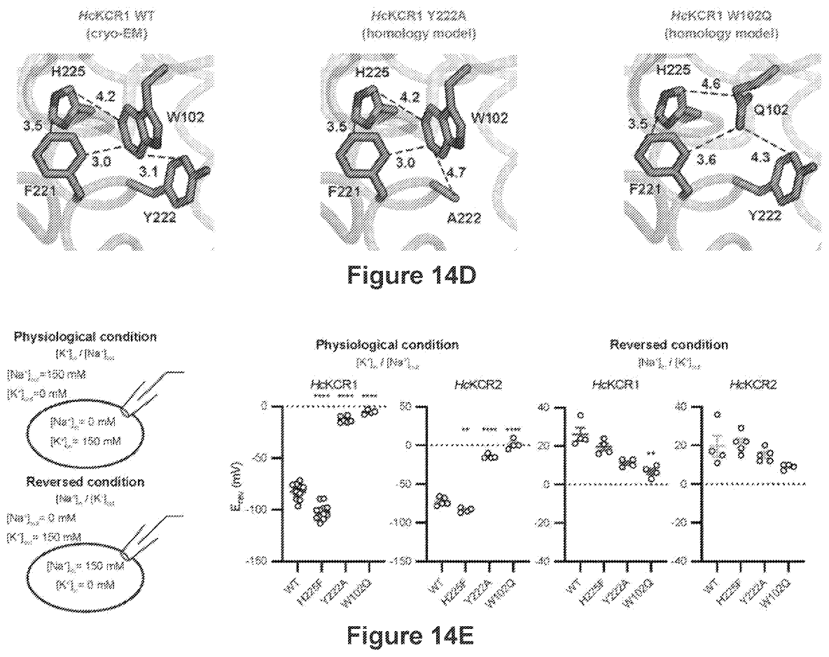

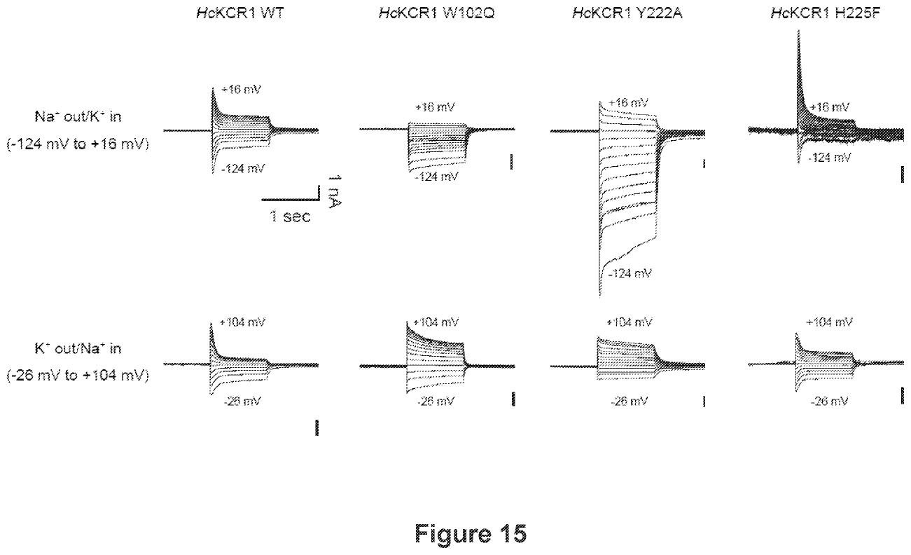

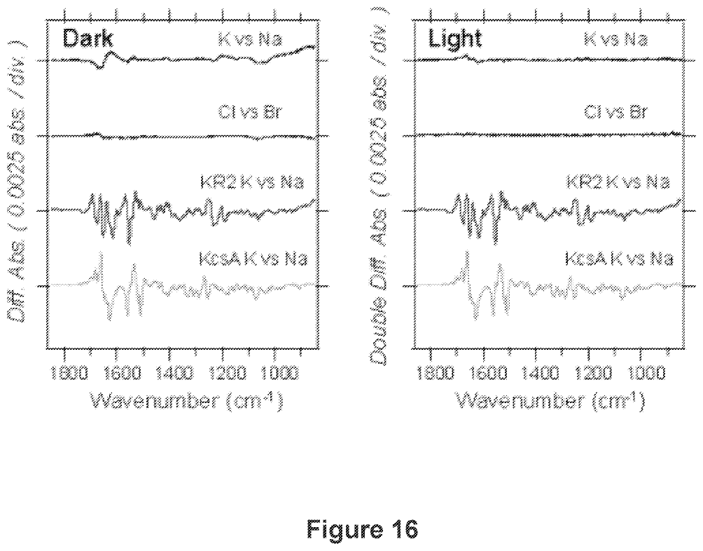

A prediction of this hypothesis would be that robust Na⁺ currents would be observed through KCRs under altered electrochemical gradient conditions: either in the form of 1) inward Na⁺ currents at membrane potentials more negative than the reversal potential for smaller (hydrated) K⁺ ions that could otherwise flow outward and outcompete larger hydrated Na⁺ ions at the size exclusion filter, or 2) outward Na⁺ currents under reversed chemical gradients of Na⁺ and K⁺, at strongly positive membrane potentials to deter competing inward K⁺ flux. These two predictions were tested, first indeed observing the predicted robust inward currents in WT KCRs in the absence of extracellular K⁺, when Vₘ was <−80 mv. To test the second prediction, electrophysiology was performed after reversing the natural electrochemical gradients of Na⁺ and K⁺, that is, imposing high extracellular K⁺ concentration ([K⁺]ₒᵤₜ) and high intracellular Na⁺ concentrations ([Na⁺]ᵢₙ) (FIG. 14E, left). WT HcKCR1 maintained robust photocurrents under these new conditions (FIG. 15) but with Eᵣₑᵥ shifted positively to 26.2±3.3 mV under these conditions (FIG. 14E, middle and right), revealing that Na⁺ indeed could efficiently move from the intracellular to the extracellular side and the proposed K⁺ selective filter could not prevent this from occurring. The same effect was observed in HcKCR1 mutants with different ion selectivity or even HcKCR2 WT and its mutants (FIG. 14E, middle and right). Consistent with this interpretation, Attenuated Total Reflection Fourier-Transform InfraRed (ATR-FTIR) spectroscopy of WT HcKCR1 showed that K⁺ does not stably bind to the selectivity filter both in dark and light conditions (FIGS. 1 and 16). These results support the idea that the HcKCR K⁺ selectivity filter does not tightly bind and specifically coordinate dehydrated K⁺ as observed in canonical K⁺ channels (FIG. 17), but instead favors flux of the smaller hydrated K⁺ ion at an aromatic size exclusion filter at the EV, and thus deters the entrance of Na⁺ ions (which would have to be chiefly from the extracellular side under physiological conditions, and which would encounter the presence of robust outward flux—and size filter occupancy—by smaller K⁺ ions).

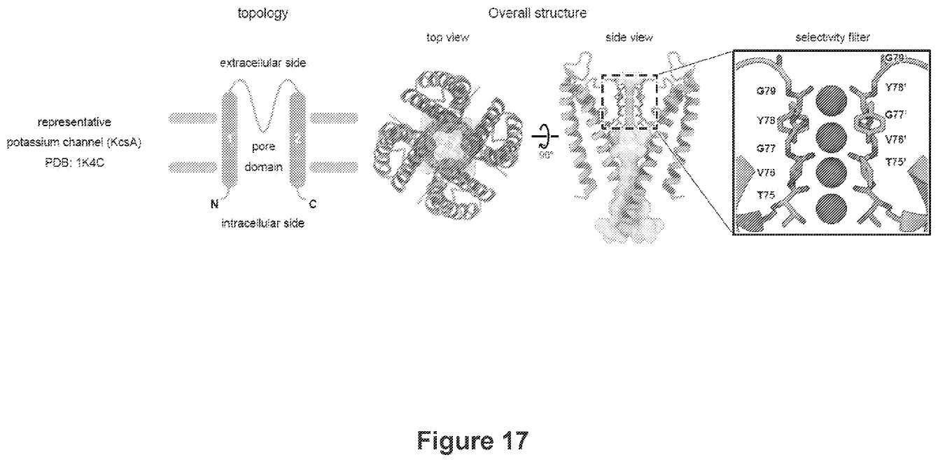

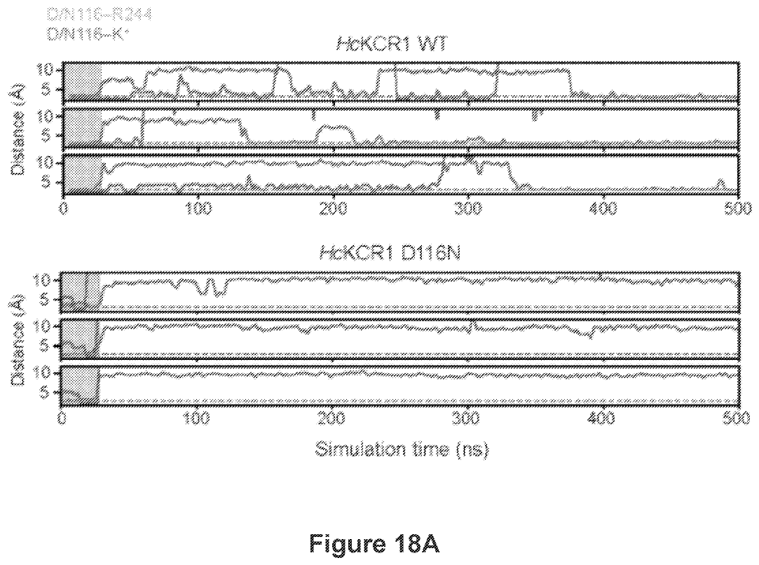

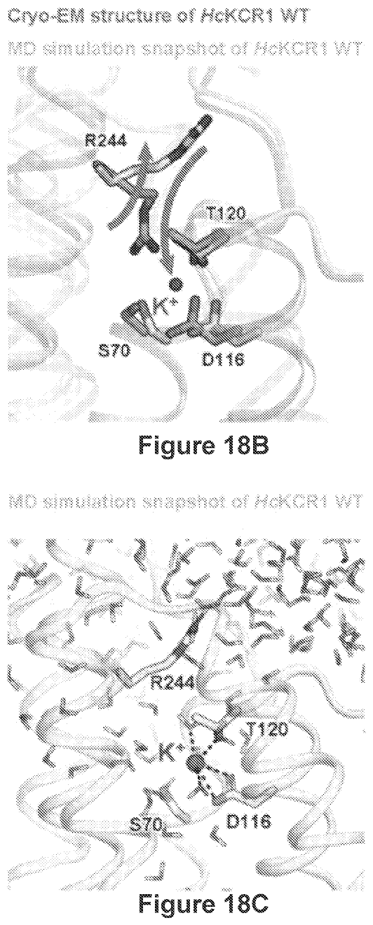

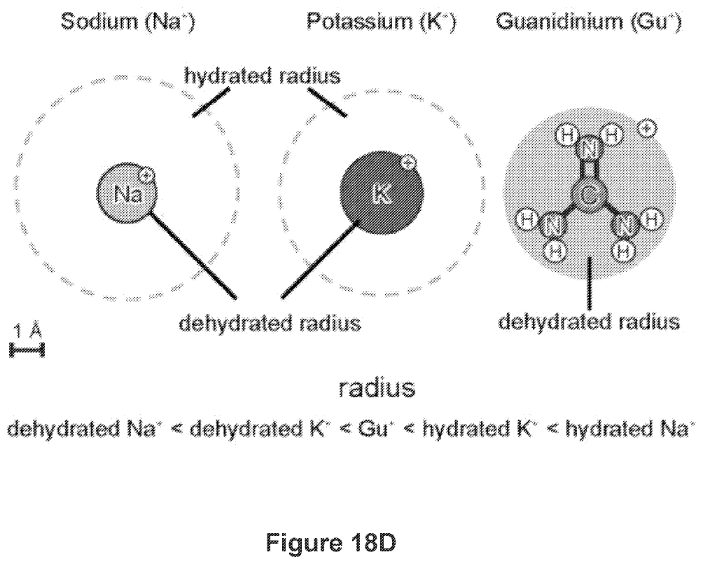

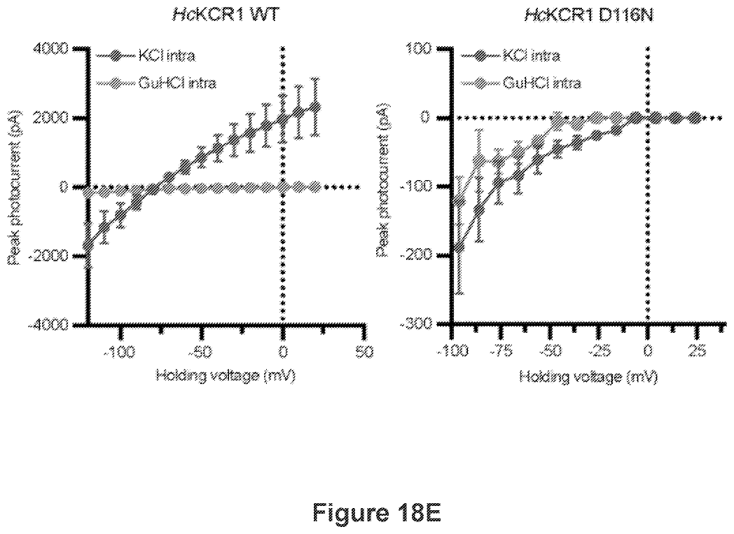

Molecular dynamics (MD) simulations of HcKCR1 were performed in the presence of K⁺. A series of 500-ns simulations provided three important findings (FIGS. 18A-18C). First, K⁺ does not stably bind at the EV but does occasionally bind spontaneously to a site near the IV, defined by the constriction formed at D116 and T120. Of note, these interactions are transient (FIG. 18A, top), consistent with the ATR-FTIR result (FIG. 16). Second, these binding events are always accompanied by a loss of the salt bridge between R244 and D116 and reorientation of the R224 side chain towards the solvent. The K⁺ essentially replaces the guanidinium group of R224, making simultaneous binding unfavorable (FIG. 18B). Third, when K⁺ binds to D116 and T120, some water molecules surrounding K⁺ in the solution are removed, rendering the K⁺ partially dehydrated (FIG. 18C). These observations suggest that D116, and surrounding residues, present a favourable environment for partial dehydration of ions entering the cavity from the intracellular side. If this hypothesis is correct, the EV selectivity filter may also encounter partially dehydrated K⁺ ions arriving via the IV, which would also be good candidates for passing through an EV aromatic size exclusion filter since partially dehydrated K⁺ ions will have even smaller radii.

Computational and functional analyses of the D116N mutant were in good agreement with this hypothesis. MD simulation of the D1116N mutant revealed that K⁺ does not bind to D116N and T120, unlike the case for WT HcKCR1 (FIG. 18A, bottom). Moreover, electrophysiology showed that currents were nearly abolished in this mutant, however, a small inward current of WT HcKCR1 remained, reversing near 0 mV in this severe loss-of-function mutant and revealing that D116 is not absolutely required for ion conduction in general (FIG. 7B).

To further examine the importance of dehydration, the effect of guanidinium ions (Gu⁺) on the channel activity of HcKCR1 was analysed (FIGS. 18D and 18E). Gu⁺ is a monovalent cation with radius larger than those of dehydrated K⁺ or Na⁺ but smaller than those of hydrated K⁺ or Na⁺ (FIG. 18D); moreover, Gu⁺ is known to be one of the most weakly hydrated cations in solution. It was found that addition of Gu⁺ to the intracellular solution completely inhibited channel activity of WT HcKCR1 (FIG. 18E, left). The lack of outward Gu⁺ current in itself indicated that either Gu⁺ acts as a pore blocker by interacting with a specific binding site in the pore, or that Gu⁺ is simply too large an ion for HcKCR1 to transport (in which case even larger cations—such as fully hydrated K⁺ or Na⁺—would also be too large for HcKCR1 to transport, suggesting that partial dehydration may be important for ion transport). It was next found that Gu⁺ indeed blocked the transport of K⁺ and Na⁺ (FIG. 18E, left). Considering the structural similarity between Gu⁺ and the side chain of the arginine residue, it is possible that D116 serves as a Gu⁺ binding site as R244 interacts with D116, and that Gu⁺ binding to this site prevents ion flux. This idea was supported by further electrophysiology of the D116N mutant showing that Gu⁺ does not significantly inhibit the inward current remaining in the mutant (FIG. 18E, right), presumably because it is no longer able to bind to D116 just as is the case with the R244 guanidinium moiety.

### Example 6—A Proposed Mechanism for the K⁺ Selectivity by KCRs

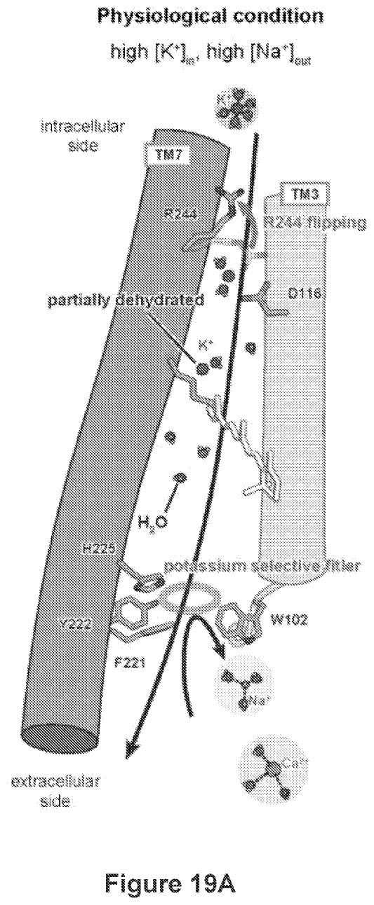

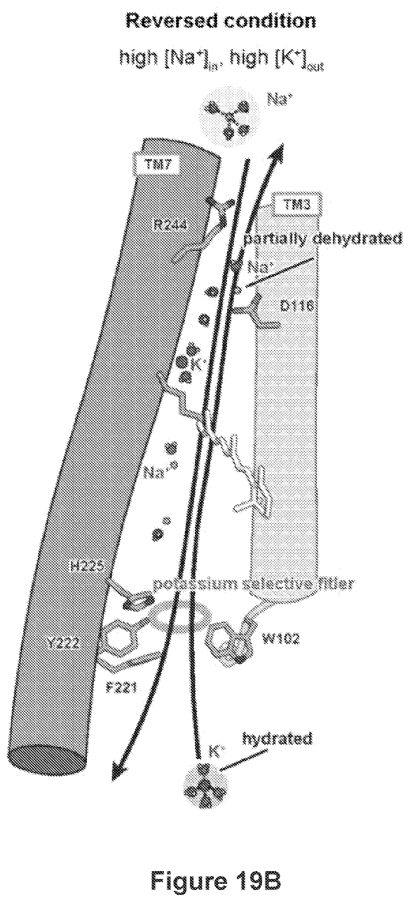

The structural, electrophysiological, spectroscopic, and computational data collectively provide insights into the mechanism for K⁺ selectivity by KCRs. Under physiological conditions, the concentrations of most simple cations, including Na⁺, Ca²⁺, and Mg²⁺, are higher on the extracellular side, while the concentration of K⁺ is higher on the intracellular side. When the KCR is opened by light, fully-hydrated Na⁺ approaching from the extracellular side encounters a barrier at the size selectivity filter formed by W102, F/Y221, Y222, and H225 (hydrated Ca²⁺ and Mg²⁺, even larger than hydrated Na⁺, would be blocked by the same mechanism); moreover hydrated Na⁺ is outcompeted by 1) smaller hydrated K⁺ ions from the extracellular side, if present, and 2) even smaller partially dehydrated K⁺ ions from the intracellular side (FIG. 19A) where R244 is mobile; when it flips out intracellular K⁺ can bind to D116, stripping away some water molecules in the hydration shell.

A similar route would also be available for intracellular Na⁺, including partial dehydration at D116, but would rarely be taken under physiological conditions due to very low [Na⁺]. However, Na⁺ currents can be observed, especially if K⁺ flux is prevented by manipulating electrochemical gradients. By completely removing extracellular K⁺ (FIG. 14E), inward Na⁺ currents were enabled at Vₘ<−80 mV (revealing a phenomenon which can be observed in canonical K⁺ channels as well, such as Kv2.1—namely K⁺ block of K⁺ channels, specifically the block of non-K⁺ flux through K⁺ channels by K⁺ ions that compete more effectively at the selectivity filter). Consistent with this interpretation, when artificially reversing the concentrations of intracellular K⁺ and extracellular Na⁺, the direction of ion flow was also reversed (FIG. 19B). Notably, presumably-hydrated K⁺ can still efficiently flow from the extracellular to intracellular side in this condition (FIGS. 18B and 15), suggesting that the size boundary for ionic species that can pass through the selectivity filter is above that of hydrated K⁺.

In conclusion, KCRs adopt a unique mechanism to specifically select for K⁺ flux, in a manner unlike canonical K⁺ channels. The KCR channels employ an aromatic size exclusion filter for hydrated or partially hydrated ions at the extracellular side, rather than specifically coordinating dehydrated ions. Species with small, hydrated radii are favored for transport, a process dominated by outward K⁺ flux under physiological conditions due to the strong electrochemical gradient in that direction.

### Example 7—Amino Acid Alignment

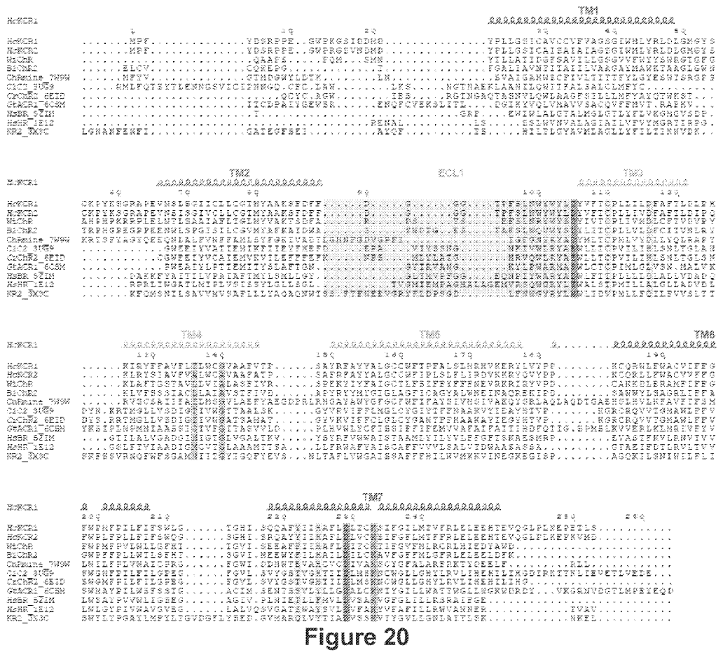

A structure-based amino acid sequence alignment of microbial rhodopsins is provided in FIG. 20. The sequences include HcKCR1 (SEQ ID NO: 1), HcKCR2 (SEQ ID NO: 2), WiChR (SEQ ID NO: 3), B1ChR2 (SEQ ID NO: 4), ChRmine (PDB: 7W9W; SEQ ID NO: 5), C1C2 (PDB: 3UG9; SEQ ID NO: 6), CrChR2 (PDB: 6EID; SEQ ID NO: 7), GtACR1 (PDB: 6CSM; SEQ ID NO: 8), HsBR (PDB: 5ZIM; SEQ ID NO: 9), HsHR (PDB: 1E12; SEQ ID NO: 10), and KR2 (PDB: 3X3C; SEQ ID NO: 11). The sequence alignment was created using PROMALS3D and ESPript 3 servers. Secondary structure elements for HcKCR1 are shown as coils. The lysine forming the Schiff base with retinal is colored in purple. The cysteine for the step-function variant is colored in green. The counterion candidates are colored in red. The ECL1 regions are highlighted in pale yellow. The residues forming the pocket for the β-ionone ring are colored in orange. The residues forming the dehydration gate and K⁺ selectivity filter are colored in cyan.

Accordingly, the preceding merely illustrates the principles of the present disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein.

What is claimed is:

1. A light gated potassium selective channelrhodopsin (KCR), comprising:
a oligomeric assembly of subunits forming a central pore, wherein each subunit comprises at least one transmembrane domain, and wherein at least one subunit comprises an amino acid substitution that alters a property of the oligomeric assembly.

2. The light gated KCR of claim 1, wherein the oligomeric assembly of subunits comprises at least two subunits.

3. The light gated KCR of claim 1 or 2, wherein the oligomeric assembly of subunits is selected from a dimer, a trimer, a pentamer, and a hexamer.

4. The light gated KCR of any one of claims 1-3, wherein each subunit comprises seven transmembrane domains, wherein each transmembrane domain forms a helix.

5. The light gated KCR of any one of claims 1-4, wherein each subunit further comprises a chromophore linked to the transmembrane domain.

6. The light gated KCR of claim 5, wherein the chromophore is linked to the transmembrane domain by a Schiff base linkage.

7. The light gated KCR of any one of claims 1-6, wherein the oligomeric assembly of subunits is derived from a pump like channelrhodopsin.

8. The light gated KCR of any one of claims 1-7, wherein the oligomeric assembly of subunits is derived from a hyphochytrid protist.

9. The light gated KCR of claim 8, wherein the hyphochytrid protist is Hyphochytrium catenoides.

10. The light gated KCR of any one of claims 1-9, wherein the oligomeric assembly of subunits is derived from Hyphochytrium catenoides Kalium channelrhodopsin 1 (HcKCR1) or Hyphochytrium catenoides Kalium channelrhodopsin 2 (HcKCR2).

11. The light gated KCR of any one of claims 1-10, wherein each subunit comprises an amino acid substitution that alters a property of the KCR.

12. The light gated KCR of claim 11, wherein the property is selected from one or more of ion selectivity and channel kinetics.

13. The light gated KCR of claim 11 or 12, wherein the property is selected from increased potassium selectivity, sodium and potassium non-selectivity, and increased τ_{off}.

14. The light gated KCR of any one of claims 11-13, wherein the amino acid substitution is selected from one or more of: W102Q, D105N, Y106W, T109A, C110T, D116N, T136A, A136T, G140A, A140G, F221A, Y222A, Y222F, H225F, H225A, H225Y, and D229N.

15. The light gated KCR of any one of claims 1-14, wherein the oligomeric assembly of subunits is derived from Hyphochytrium catenoides Kalium channelrhodopsin 1 (HcKCR1) or Hyphochytrium catenoides Kalium channelrhodopsin 2 (HcKCR2), and further comprises an amino acid substitution is selected from one or more of C110T, Y222A, and H225F.

16. A nucleic acid encoding a light gated KCR of any one of claims 1-15.

17. The nucleic acid of claim 16, further comprising a promoter.

18. The nucleic acid of claim 16, wherein the promoter is selected from an inducible promoter and a constitutive promoter.

19. The nucleic acid of any one of claims 16-18, wherein the coding sequence is codon optimized.

20. A vector comprising the nucleic acid the nucleic acid of any one of claims 16-19.

21. The vector of claim 20, wherein the vector is selected from a replication vector, a transfection vector, and an expression vector.

22. The vector of claim 20 or 21, wherein the vector is selected from a plasmid, a cosmid, a virion, a viroid, a virus, and a bacterium.

23. A method of expressing a light gated potassium selective channelrhodopsin (KCR), the method comprising:
contacting a cell with a nucleic acid of any one of claims 16-19 or a vector of any one of claims 20-22.

24. A cell expressing the light gated KCR of any one of claims 1-15.

25. The cell of claim 24 comprising the nucleic acid of any of one claims 16-19.

26. The cell of claim 24 or 25, wherein the cell is a neuron.

27. A method of optogenetically controlling a cell, the method comprising:
illuminating the cell of claim 24 or 25 with a light encompassing a wavelength including the absorbance maximum of the light gated KCR of any one of claims 1-15, wherein illuminating the cell triggers transport of an ion into the cell.
